SEQUENCE LISTING (66 sequences)

SEQ ID NO: 1 - DNA, 19 bp, synthetic construct
  source 1..19: mol_type = other DNA
cgtgaccaccctgacctgg

SEQ ID NO: 2 - DNA, 20 bp, synthetic construct
  source 1..20: mol_type = other DNA
gtggcggatcttgaagttgg

SEQ ID NO: 3 - DNA, 20 bp, synthetic construct
  source 1..20: mol_type = other DNA
tgcagtgcttcagccgctac

SEQ ID NO: 4 - DNA, 20 bp, synthetic construct
  source 1..20: mol_type = other DNA
ccagcaggaccatgtgatcg

SEQ ID NO: 5 - DNA, 26 bp, synthetic construct
  source 1..26: mol_type = other DNA
ctcagttcttaggcttcagcaattac

SEQ ID NO: 6 - DNA, 24 bp, synthetic construct
  source 1..24: mol_type = other DNA
ccaaaggcaagatatgatttagga

SEQ ID NO: 7 - DNA, 24 bp, synthetic construct
  source 1..24: mol_type = other DNA
tgtccgtggttgccaaggataaca

SEQ ID NO: 8 - DNA, 24 bp, synthetic construct
  source 1..24: mol_type = other DNA
aatccgggtacctgcttgtgagtt

SEQ ID NO: 9 - DNA, 21 bp, synthetic construct
  source 1..21: mol_type = other DNA
ccacggttagcaccaaatagc

SEQ ID NO: 10 - DNA, 23 bp, synthetic construct
  source 1..23: mol_type = other DNA
cacacagaacctctgccctactc

SEQ ID NO: 11 - DNA, 19 bp, synthetic construct
  source 1..19: mol_type = other DNA
ctgggaagggtctacccac

SEQ ID NO: 12 - DNA, 20 bp, synthetic construct
  source 1..20: mol_type = other DNA
ggtgctatgttagcggcctc

SEQ ID NO: 13 - DNA, 21 bp, synthetic construct
  source 1..21: mol_type = other DNA
tgaagggccagtgtgaaaagt

SEQ ID NO: 14 - DNA, 25 bp, synthetic construct
  source 1..25: mol_type = other DNA
ttgaacagtccacagacaagatctc

SEQ ID NO: 15 - DNA, 20 bp, synthetic construct
  source 1..20: mol_type = other DNA
ccaggatcagcagtgtgaga

SEQ ID NO: 16 - DNA, 20 bp, synthetic construct
  source 1..20: mol_type = other DNA
tgtttgtggtgttgtagcgg

SEQ ID NO: 17 - DNA, 23 bp, synthetic construct
  source 1..23: mol_type = other DNA
ggtctgagagcccgaactctcct

SEQ ID NO: 18 - DNA, 23 bp, synthetic construct
  source 1..23: mol_type = other DNA
cccatgtctgggcacctctcttt

SEQ ID NO: 19 - DNA, 20 bp, synthetic construct
  source 1..20: mol_type = other DNA
cacttctggagggagagtgg

SEQ ID NO: 20 - DNA, 20 bp, synthetic construct
  source 1..20: mol_type = other DNA
atgcctggaagaaagaccga

SEQ ID NO: 21 - DNA, 18 bp, synthetic construct
  source 1..18: mol_type = other DNA
aagggctccatgggattc

SEQ ID NO: 22 - DNA, 18 bp, synthetic construct
  source 1..18: mol_type = other DNA
acgactggaagccggaac

SEQ ID NO: 23 - DNA, 19 bp, synthetic construct
  source 1..19: mol_type = other DNA
ccgcaaccacatccacgta

SEQ ID NO: 24 - DNA, 20 bp, synthetic construct
  source 1..20: mol_type = other DNA
cggaccgccagtgagttatc

SEQ ID NO: 25 - DNA, 20 bp, synthetic construct
  source 1..20: mol_type = other DNA
cagcgcttctcagtcagtgt

SEQ ID NO: 26 - DNA, 20 bp, synthetic construct
  source 1..20: mol_type = other DNA
ctgctgttccaggtcagaga

SEQ ID NO: 27 - DNA, 22 bp, synthetic construct
  source 1..22: mol_type = other DNA
tcttccccaacggtactatcca

SEQ ID NO: 28 - DNA, 19 bp, synthetic construct
  source 1..19: mol_type = other DNA
cgaagcggctgtggtcttt

SEQ ID NO: 29 - DNA, 20 bp, synthetic construct
  source 1..20: mol_type = other DNA
ttgcctgatgatgttttcca

SEQ ID NO: 30 - DNA, 20 bp, synthetic construct
  source 1..20: mol_type = other DNA
ctggccctcaacttcttcag

SEQ ID NO: 31 - DNA, 20 bp, synthetic construct
  source 1..20: mol_type = other DNA
tggggacctcagaacccttg

SEQ ID NO: 32 - DNA, 19 bp, synthetic construct
  source 1..19: mol_type = other DNA
ggctgcatttggggaagtg

SEQ ID NO: 33 - DNA, 23 bp, synthetic construct
  source 1..23: mol_type = other DNA
ccccaatctcgatatgtttgatg

SEQ ID NO: 34 - DNA, 23 bp, synthetic construct
  source 1..23: mol_type = other DNA
ttgacacactctctgccttctga

SEQ ID NO: 35 - DNA, 21 bp, synthetic construct
  source 1..21: mol_type = other DNA
gggtgcgttttaactgccata

SEQ ID NO: 36 - DNA, 20 bp, synthetic construct
  source 1..20: mol_type = other DNA
gcccaaacccaagtcagtga

SEQ ID NO: 37 - DNA, 20 bp, synthetic construct
  source 1..20: mol_type = other DNA
gaggagccgacgttcataga

SEQ ID NO: 38 - DNA, 20 bp, synthetic construct
  source 1..20: mol_type = other DNA
agcactgacggtttagctct

SEQ ID NO: 39 - DNA, 20 bp, synthetic construct
  source 1..20: mol_type = other DNA
gcagtacagccccaaaatgg

SEQ ID NO: 40 - DNA, 21 bp, synthetic construct
  source 1..21: mol_type = other DNA
ggtccttttcaccagcaagct

SEQ ID NO: 41 - DNA, 20 bp, synthetic construct
  source 1..20: mol_type = other DNA
ggaggagatcagggtcctgg

SEQ ID NO: 42 - DNA, 19 bp, synthetic construct
  source 1..19: mol_type = other DNA
ctaggatggtctgcctggg

SEQ ID NO: 43 - DNA, 20 bp, synthetic construct
  source 1..20: mol_type = other DNA
gctgctactgatgctcctgg

SEQ ID NO: 44 - DNA, 19 bp, synthetic construct
  source 1..19: mol_type = other DNA
ctagacccgcagcttcctc

SEQ ID NO: 45 - DNA, 23 bp, synthetic construct
  source 1..23: mol_type = other DNA
cctcgatgtgaaattcctgaaca

SEQ ID NO: 46 - DNA, 18 bp, synthetic construct
  source 1..18: mol_type = other DNA
tcctgggcgtcctttacg

SEQ ID NO: 47 - DNA, 20 bp, synthetic construct
  source 1..20: mol_type = other DNA
ccagttggagacgacctaca

SEQ ID NO: 48 - DNA, 20 bp, synthetic construct
  source 1..20: mol_type = other DNA
gtggcagagtttgcactcaa

SEQ ID NO: 49 - DNA, 20 bp, synthetic construct
  source 1..20: mol_type = other DNA
ggctcctggacactacacct

SEQ ID NO: 50 - DNA, 20 bp, synthetic construct
  source 1..20: mol_type = other DNA
catagttggcctccaccact

SEQ ID NO: 51 - DNA, 22 bp, synthetic construct
  source 1..22: mol_type = other DNA
gccctagaaggtgctggtttgg

SEQ ID NO: 52 - DNA, 23 bp, synthetic construct
  source 1..23: mol_type = other DNA
gtggggtcacctcagtgagtagg

SEQ ID NO: 53 - DNA, 26 bp, synthetic construct
  source 1..26: mol_type = other DNA
gcagtctagaattgttcagagccaga

SEQ ID NO: 54 - DNA, 26 bp, synthetic construct
  source 1..26: mol_type = other DNA
tccaaactctttatgaactgctcgtg

SEQ ID NO: 55 - DNA, 21 bp, synthetic construct
  source 1..21: mol_type = other DNA
ccaaaatcagcaaaccccatt

SEQ ID NO: 56 - DNA, 20 bp, synthetic construct
  source 1..20: mol_type = other DNA
ctggatcccaggttcgaaac

SEQ ID NO: 57 - DNA, 19 bp, synthetic construct
  source 1..19: mol_type = other DNA
agcgagaagaggccgtttg

SEQ ID NO: 58 - DNA, 21 bp, synthetic construct
  source 1..21: mol_type = other DNA
tcagtaccagagtctgagtcg

SEQ ID NO: 59 - DNA, 22 bp, synthetic construct
  source 1..22: mol_type = other DNA
aagaaagaccaccccgattaca

SEQ ID NO: 60 - DNA, 21 bp, synthetic construct
  source 1..21: mol_type = other DNA
cagcgccttgaagatagcatt

SEQ ID NO: 61 - DNA, 20 bp, synthetic construct
  source 1..20: mol_type = other DNA
gacgtcggagctctctgctt

SEQ ID NO: 62 - DNA, 20 bp, synthetic construct
  source 1..20: mol_type = other DNA
gccttctgcatagccacctc

SEQ ID NO: 63 - DNA, 22 bp, synthetic construct
  source 1..22: mol_type = other DNA
ttgaatgcatgacctggaatca

SEQ ID NO: 64 - DNA, 25 bp, synthetic construct
  source 1..25: mol_type = other DNA
ttccctttaaggtagctcctaggtt

SEQ ID NO: 65 - DNA, 20 bp, synthetic construct
  source 1..20: mol_type = other DNA
cccctgaagagcatccgaca

SEQ ID NO: 66 - DNA, 19 bp, synthetic construct
  source 1..19: mol_type = other DNA
tggcggggacattgaaggt

(19) United States
(12) Patent Application Publication
(10) Pub. No.: US 2026/0347791 A1
(43) Pub. Date: Nov. 5, 2026

(54) TESTICULAR ORGANOIDS, METHODS OF PREPARING THE SAME AND USES THEREOF

(71) Applicant: BAR-ILAN UNIVERSITY, Ramat Gan (IL)

(72) Inventors: Nitzan GONEN, Hod Hasharon (IL); Aviya STOPEL, Givat Shmuel (IL)

(21) Appl. No.: 19/698,305

(22) Filed: Jun. 4, 2026

Related U.S. Application Data
(63) Continuation of application No. PCT/IL2024/051198, filed on Dec. 18, 2024.
(60) Provisional application No. 63/612,384, filed on Dec. 20, 2023.

(51) Int. Cl. C12N 5/071

(52) U.S. Cl. CPC C12N 5/0683; C12N 2501/11; C12N 2501/119; C12N 2501/16; C12N 2501/30; C12N 2501/31; C12N 2501/392

(57) ABSTRACT

Provided herein are testicular organoids generated from primary embryonal or neonatal cells, methods for their preparation, and uses thereof, wherein the organoids are capable of generating tubule-like structures and cellular organization resembling that of the in vivo testis and exhibit a gene expression profile that recapitulates that observed in in vivo testis.

### CROSS REFERENCE TO RELATED APPLICATIONS

This application is a Bypass Continuation of PCT Patent Application No. PCT/IL2024/051198 having International filing date of Dec. 18, 2024, which claims the benefit of priority of U.S. Provisional Patent Application No. 63/612,384, filed Dec. 20, 2023, the contents of which are all incorporated herein by reference in their entirety.

### REFERENCE TO AN ELECTRONIC SEQUENCE LISTING

The contents of the electronic sequence listing (BRD-004-PCT.xml; Size: 59,119 bytes; and Date of Creation: Dec. 16, 2024) is herein incorporated by reference in its entirety.

### FIELD OF THE INVENTION

The present invention relates to testicular organoids, generated from embryonal or neonatal primary cells, methods for their preparation and uses thereof.

### BACKGROUND OF THE INVENTION

The testes are part of the male reproductive system and are responsible for production, storage, and maturation of spermatids, as well as androgen production and secretion throughout life [1, 2]. In the mouse, testes develop at embryonic day (E) 11.5 from the bipotential gonad upon the expression of the sex determining gene, Sry, and its downstream target gene, Sox9 [3, 4]. The testis is composed of two main compartments: the testis cords and the interstitium. The testis cords are comprised of the somatic Sertoli cells that encapsulate the germ cells (gonocytes). Peritubular Myoid Cells (PM) are located outside the testis cord, and together with Sertoli cells, help to establish the testicular basement membrane, mainly composed of extra cellular matrix (ECM) proteins. Within the interstitium lie the steroidogenic Leydig cells that secrete androgens, as well as endothelial cells, immune cells, and progenitor cells [1, 5]. At puberty, Sertoli cells transform from an immature-to-mature cell state, and the testis cords become the seminiferous tubules. During this stage, the gonocytes, which were initially located at the center of the testis cords, migrate to the periphery of the tubules, next to the basement membrane, and are now termed spermatogonial stem cells (SSC). SSCs which reside within their niche are the source of spermatogenesis in adulthood throughout life [6]. Sertoli cells are the only somatic cells being in direct contact with the germ cells, and their roles in nurturing and providing the signals and support needed for germ cell proliferation, maturation, and spermatogenesis are well established [1-3]. Sertoli cells are considered to be the “organising hub” of the testis at both embryonic and adult stages [1-3].

Dysfunction of testis development or function leads to various diseases including Disorders of Sex Development (DSD) and infertility. DSD is defined as discordance between the genetic, gonadal, and anatomical sex of an individual with a prevalence of 1:4000 newborns [7, 8]. While many genes and pathways have been shown to be involved in DSD, currently only 50% of DSD cases receive a genetic diagnosis following whole exome sequencing (WES). Infertility is defined by the failure to achieve a pregnancy after 12 months or more of regular unprotected sexual intercourse. It is experienced by 1 in 6 people [9], and male infertility accounts for ~50% of cases. While male infertility is easily identified and classified into sub-groups, very little is known about the genetic and environmental mechanisms leading to male infertility [10].

Currently, there is no proper in vitro system to model the testis at embryonic or adult stages. Culture of primary testicular cells in 2D settings and serum-containing media leads to loss of their normal characteristics, and gene expression patterns rapidly diverge from normal testis [11]. Additionally, there are no reliable testicular cell lines that recapitulate the in vivo testicular cells [12]. This limits studying testis development to in vivo models in mice, where the process can be long, expensive, and laborious. Furthermore, while mouse and human sex determination and testicular function systems are generally similar, they are not fully conserved, and many DSD and infertility cases cannot be successfully modelled in mice [13, 14].

Organoids are three dimensional (3D) structures, cultured in vitro, that closely resemble the structure and function of true organs [15]. Organoids have been developed to model multiple types of organs such as the intestine, brain, kidney, retina, and others [15]. In recent years, several studies described the generation of testicular organoids, mostly from primary testicular cells of human, mouse, pig, and rat (reviewed in [16, 17]). These studies used different approaches for culturing the primary testicular cells including decellularized testis, Matrigel, soft agar, as well as microfluidic devices [18-23]. While some models were able to recapitulate testis structure and formation of tubule like-structure, preservation of germ cells, and even mild entry to meiosis, most failed to show preservation of the organoids for prolonged periods and did not assess gene expression profiles in comparison to in vivo testis.

Thus, there is an unmet need for an efficient, functional testicular organoid that is produced from primary cells and can be used to model native testis structure and function and further be used as a platform to explore normal testis development and associated pathologies.

### SUMMARY OF THE INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

According to some embodiments, there are provided herein testicular organoid systems, methods for their generation and uses thereof, wherein the organoids are generated from primary testis cells, and exhibit structural and functional traits comparable to that of a native (in-vivo) testis.

According to some embodiments, advantageously, the testicular organoids disclosed herein are generated from non-immortalized cells, in particular, from neonatal mouse primary testicular cells and/or from embryonal cells. Surprisingly, the organoids can generate tubule-like structures and cellular organization resembling that of the in vivo testis. Moreover, surprisingly, as demonstrated herein, the gene expression profile of the organoids demonstrate a profile that recapitulates that observed in in vivo (native) testis.

According to some embodiments, embryonic testicular cells and/or neonatal testicular cells, but not adult testicular cells are capable of forming organoids.

According to some embodiments, surprisingly, as demonstrated herein below, the organoids can be maintained in culture for at least 3 weeks, at least 4 weeks, at least 7 weeks, at least 8 weeks, and further exhibit signs of entry into meiosis.

According to some embodiments, there are further provided herein culture media compositions, that can specifically promote immature versus mature Sertoli cell and Leydig cell states, thereby facilitating organoid maturation in vitro.

According to some embodiments, the generated organoids can be used as model system for research of testes development and function, including translational applications for elucidation and treatment of developmental sex disorders (DSD) and infertility.

According to some embodiments, there is provided a testis organoid including primary testis cells, obtained from neonatal and/or embryonic testis, wherein the cells form a 3D structure on a transwell insert in the presence of a defined culture media, said organoid is viable for at least three weeks in culture and capable of expressing one or more cellular markers at a spatial and/or temporal pattern similar to that of an in-vivo testis.

According to some embodiments, the organoid may include at least about 1×10⁵ primary testis cells.

According to some embodiments, the testis cells include Sertoli cells, Leydig cells, peritubular myoid (PM) cells and gonocytes.

According to some embodiments, the organoid is viable for at least 7 weeks in culture.

According to some embodiments, the Sertoli cells cellular markers may include Sox9, Nr5a1, Wt1, Clu, Ar, Amh, Fshr, Gdnf, Gata4, Ptgds, Gata1, or any combinations thereof.

According to some embodiments, the Leydig cells cellular markers may include 3ßHsd, StAR, Ins13, Cyp1 1a1, or any combinations thereof.

According to some embodiments, the PM cells cellular markers include α-Sma.

According to some embodiments, the gonocytes cellular markers include Plzf, Scyp3, DDX4, or any combinations thereof.

According to some embodiments, the organoid includes tubular-like structures comprising Sertoli cells and interstitial regions including Leydig cells, located externally to said tubular structure, after 7 or more days in culture.

According to some embodiments, the tubular-like structures are organized spatially to mimic seminiferous tubules of in vivo testis.

According to some embodiments, the organoid is capable of expressing acrosin, after at least 7 days in culture.

According to some embodiments, at least some spermatogonial stem cells (SSC) of the organoid can enter meiosis, after at least 7 days in culture.

According to some embodiments, the organoid is capable of supporting entry of spermatogonial stem cells (SSC) into meiosis and maintain germ cell function for at least 7 weeks in vitro.

According to some embodiments, the defined culture media is a serum-free media, comprising B-27 supplement and one or more of. Epidermal Growth Factor (EGF), Follicular stimulating hormone (FSH), Fibroblast Growth Factor 9 (FGF9), Prostaglandin D2 (PDG2), Testosterone and Activin A. Each possibility is a separate embodiment.

According to some embodiments, the neonatal cells are obtained from neonatal mice, aged between postnatal days 4-7 (P4-P7).

According to some embodiments, the embryonal cells are obtained from mice embryos on embryonic days E 12.5-14.5.

According to some embodiments, the testis may be used for modelling testis-related pathologies and/or drug testing.

According to some embodiments, there is provided a method for producing a testis organoid, the method includes one or more of the steps of:
a) obtaining primary testis cells from a neonatal and/or embryonic testis;
b) seeding the testis cells on a Transwell insert;
c) culturing the cells in the presence of a defined culture medium including B-27 supplement;
   wherein the organoid is viable for at least three weeks in culture and is capable of expressing one or more cellular markers at a spatial and/or temporal pattern similar to that of an in-vivo testis.

According to some embodiments, the method includes seeding at least about 5×10⁴ testis cells per organoid.

According to some embodiments, the method includes seeding at least about 2×10⁵ testis cells per organoid.

According to some embodiments, the testis cells are obtained by mechanical and/or enzymatic dissociation of the testis.

According to some embodiments, the primary testis cells include Sertoli cells, Leydig cells, peritubular myoid (PM) cells and gonocytes.

According to some embodiments, the defined culture media is serum-free media.

According to some embodiments, the defined culture media may include: Epidermal Growth Factor (EGF), Follicular stimulating hormone (FSH), Fibroblast Growth Factor 9 (FGF9), Prostaglandin D2 (PDG2), Testosterone, Activin A, or any combinations thereof. Each possibility is a separate embodiment.

According to some embodiments, the EGF may be added at a concentration of about 1-100 ng/ml.

According to some embodiments, the FSH may be added at a concentration of about 1-100 ng/ml.

According to some embodiments, the FGF9 may be added at a concentration of about 1-100 ng/ml.

According to some embodiments, the PDG2 may be added at a concentration of about 100-1000 ng/ml.

According to some embodiments, the Testosterone may be added at a concentration of about 0.5-2 μM.

According to some embodiments, the Activin may be added at a concentration of about 1-100 ng/ml.

According to some embodiments, the neonatal cells may be obtained from neonatal mice, aged between postnatal days 4-7 (P4-P7). According to some embodiments, the embryonal cells may be obtained from mice embryos on embryonic days E 12.5-14.5.

According to some embodiments, the Sertoli cells cellular markers may include Sox9, Nr5a1, Wt1, Clu, Ar, Amh, Fshr, Gdnf, Gata4, Ptgds, Gata1, or any combinations thereof. According to some embodiments, the

According to some embodiments, the Leydig cells cellular markers may include 3ßHsd, StAR, Ins13, Cyp11a1, or any combinations thereof. According to some embodiments, the According to some embodiments, the PM cells cellular markers include α-Sma.

According to some embodiments, the gonocytes cellular markers include Plzf, Scyp3, DDX4, or any combinations thereof.

According to some embodiments, the at least 7 days in culture, the organoid includes tubular-like structures including Sertoli cells and interstitial regions including Leydig cells, located externally to said tubular structure.

According to some embodiments, the organoid is viable for at least 7 weeks in culture.

According to some embodiments, there is provided a testis organoid generated by the method disclosed herein.

According to some embodiments, there is provided a method for producing a mature testis organoid, the method includes:
a) obtaining primary testis cells from a neonatal and/or embryonic testis;
b) seeding the testis cells on a Transwell insert;
c) culturing the cells in the presence of a first culture medium for a first period of time;
d) replacing the first cellular medium to a second culture medium; and
d) culturing the cells in the presence of the second culture medium;
   wherein the mature organoid is viable for at least three weeks in culture and is capable of expressing one or more cellular markers at a spatial and/or temporal pattern similar to that of an in-vivo testis.

According to some embodiments, the method includes seeding at least about 5×10⁴ testis cells per organoid.

According to some embodiments, the testis cells are obtained by mechanical and/or enzymatic dissociation of the testis.

According to some embodiments, the primary testis cells comprise Sertoli cells, Leydig cells, peritubular myoid (PM) cells and gonocytes.

According to some embodiments, the first culture medium is an immature culture medium configured to maintain the cells at an immature state, to allow proliferation of the cells.

According to some embodiments, the first culture medium is a serum free medium including B27 supplement and further includes: Epidermal Growth Factor (EGF), Follicular stimulating hormone (FSH), Fibroblast Growth Factor 9 (FGF9), Activin A, IGF-1, or any combinations thereof. Each possibility is a separate embodiment.

According to some embodiments, the second culture medium is mature culture medium configured to promote maturation of at least Sertoli and/or Leydig cells.

According to some embodiments, the second culture medium is a serum free medium, including B27 supplement and further includes one or more of. Epidermal Growth Factor (EGF), Follicular stimulating hormone (FSH), Testosterone, Activin A, Thyroid hormone, and Retinoic acid. Each possibility is a separate embodiment.

According to some embodiments, the neonatal cells may be obtained from neonatal mice, aged between postnatal days 4-7 (P4-P7). According to some embodiments, the embryonal cells may be obtained from mice embryos on embryonic days E 12.5-14.5.

According to some embodiments, the Sertoli cells cellular markers may include Sox9, Nr5a1, Wt1, Clu, Ar, Amh, Fshr, Gdnf, Gata4, Ptgds, Gata1, or any combinations thereof. Each possibility is a separate embodiment.

According to some embodiments, the Leydig cells cellular markers may include 3ßHsd, StAR, Ins13, Cyp11a1, or any combinations thereof. Each possibility is a separate embodiment.

According to some embodiments, the PM cells cellular markers may include α-Sma. According to some embodiments, gonocytes cellular markers comprise Plzf, Scyp3, DDX4, or any combinations thereof. Each possibility is a separate embodiment.

According to some embodiments, in presence of the first culture media, increased expression of one or more of the cellular markers Amh, Gata4 and/or Cx43 is facilitated.

According to some embodiments, in the presence of the second culture media, increased expression of Gata1 is facilitated.

According to some embodiments, the first period of time is in the range of about 4-14 days.

According to some embodiments, after at least 7 days in culture, the organoid includes tubular-like structures comprising Sertoli cells and interstitial regions comprising Leydig cells, located externally to said tubular structure.

According to some embodiments, the method may include maintaining the cultured cells under conditions conducive to the formation of a mature organoid comprising mature Sertoli cells, mature Leydig cells and tubular structures, wherein said mature organoid comprises a spatial organization resembling an in-vivo testis.

According to some embodiments, there is provided a mature testis organoid generated by the method disclosed herein.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIGS. 1A-E—Establishment of primary neonatal testicular organoids on transwell inserts. FIG. 1A shows a Schematic representation of an experimental design. Primary testicular cells from P4-P7 Sox9-IRES-GFP or TESCO-CFP pups were dissociated into single cells and re-assembled under various culturing methods and media compositions. (Scheme created using BioRender). FIG. 1B shows representative bright-field (BF) and fluorescent images of testicular organoids, from Sox9-IRES-GFP mice, cultured for 7, 14 and 21 days on transwell inserts. Scale bars, 100 m. FIG. 1C shows Representative BF and fluorescent images of the organoids forming over the first 4 days of culture. Scale bars, 100 m. FIG. 1D shows Representative BF images of organoids cultured for prolonged times. Scale bars, 500 m. FIG. 1E shows a graph of organoids area (mm²) over time in culture. Data are presented as mean area±SEM. All areas are compared to the area of D7 organoids. *P<0.05, **P<0.01, ***P<0.001, and ****P<0.0001, ns—not significant. N=3-4;

FIGS. 2A-D—Comparison of gene expression profile of cultured organoids, neonatal and adult testes. FIGS. 2A-D show bar graphs of Quantitative RT-PCR performed on mRNA extracted from neonatal or adult testes, or TESCO-CFP organoids cultured for the indicated number of days assessing Sertoli cell markers (FIG. 2A), Leydig cell markers (FIG. 2B) or other testicular cells markers (FIGS. 2C-D). Gene names are indicated. D denotes culture day of organoids, grey. P denotes days postpartum (dpp) of in vivo testis, black. Data are presented as mean 2^{-ΔΔct} values±SEM normalized to the housekeeping gene Hprt. *P<0.05, **P<0.01, ***P<0.001, and ****P<0.0001, ns—not significant. N=3-12;

FIGS. 3A-B—Testicular organoids present tubular structures and preserve main gonadal cell types. FIG. 3A shows images of whole mount co-immunostaining of markers for major types of testicular cells in organoids cultured for 21 days (“D21 organoid”). SOX9, AMH, CLD11 (Sertoli cells), DDX4 (Gonocytes), 3BHSD (Leydig cells), α-SMA (PM cells). Images at right hand columns are merged views of all channels. Scale bars, 100 m. FIG. 3B shows images of whole mount immunostaining of markers for the major types of testicular cells in organoids cultured for 21 days (“D21 organoid”) compared to sections of P28 testis. Scale bars, 100 μm;

FIGS. 4A-B—Establishment of organoid culture from embryonic testes. FIG. 4A shows representative BF and fluorescent images of organoids harvested from E12.5-E14.5 embryonic testes and cultured for 14 days (“D14 organoid”). The two left hand images (TESCO-CFP) were cultured on defined media and the two right hand images (Sox9-IRES-GFP) were cultured on immature media. Scale bars, 500 m. FIG. 4B shows images of whole mount immunostaining of markers for the major types of testicular cells in embryonic organoids cultured for 14 days. SOX9 (Sertoli cells), TRA98 (Gonocytes), 3BHSD (Leydig cells). Scale bars, 100 m;

FIGS. 5A-E—In vitro maturation of neonatal organoids in different media compositions. FIG. 5A shows a schematic representation of pre-pubertal (left), and adult testis (right). Testicular cords of the pre-pubertal testis are surrounded by peritubular myoid and Leydig cells in the interstitial compartment. Gonocytes are located at the center of the cords, surrounded by immature Sertoli cells. Post puberty and in adulthood, seminiferous tubules are surrounded by peritubular myoid cells and mature Leydig cells. Germ cells, known as spermatogonial stem cells (SSC) at this point, are situated at the basal side of the tubules, near the basement membrane. As SSC differentiate and undergo meiosis, they migrate from the basal side to the luminal side of the seminiferous tubules. Scheme created using BioRender. FIG. 5B—a schematic representation preparation of 3D organoids—Primary testicular cells from P4-P7 Sox9-IRES-GFP or TESCO-CFP mice were dissociated into single cells, re-assembled on transwell inserts and cultured in either immature or mature2 media for 21 days. A “transition” protocol was also employed wherein organoids were cultured for the first 10 days in immature media and then transitioned to mature 2 media for an additional 11 days, for a total culture period of 21 days. images created using BioRender. FIG. 5C shows representative BF images of organoids cultured with immature or mature2 media, or transition protocol, for 21 days, wherein the images were obtained at day 7 (D7), day 14 (D14) and day 21 (D21). Scale bars, 500 m. FIG. 5D shows bar graphs of organoids area (mm²) over time of organoids cultured with immature, mature 2 or transition protocol for 21 days, at the indicated time points. Data is presented as mean area±SEM. *P<0.05, ****P<0.0001, ns—not significant. N=9-14. FIG. 5E shows bar graphs of Quantitative PCR analysis of Sertoli cell markers (Amh, Cx43 and Sox9) in organoids cultured over time with immature, mature 2 or transition protocol for 21 days, compared to P5, P20 and P90 in vivo testis. Data are presented as mean 2^{-ΔΔCt} values±SEM normalized to the housekeeping gene Hprt.*P<0.05, **P<0.01, ***P<0.001, and ****P<0.0001, ns—not significant. N=3-6;

FIGS. 6A-E—SSC enter meiosis in vitro within testis organoids. FIG. 6A shows images of whole mount immunostaining of γH2AX, a marker for meiotic cells, and DDX4, which marks all germ cells in D21 organoid cultured in defined media. Scale bars, 100 m. Insets are magnified views of the boxed region. FIG. 6B shows bar graphs of Quantitative PCR analysis of Acrosin in testicular organoids over time, cultured in defined media, compared to P5, P28 and P90 in vivo testis. Data are presented as mean 2^{-ΔΔCt} values±SEM normalized to the housekeeping gene Hprt. *P<0.05, **P<0.01, ***P<0.001, ns—not significant. N=3-6. FIG. 6C shows images of whole mount immunostaining of γH2AX and DDX4 on D7 and D42 organoids cultured on transition media. White arrows indicate co-staining of γH2AX and DDX4. Scale bars, 100 m. FIG. 6D shows images of Immunostaining of testicular sections of 5, 28, 90 dpp mice. Sections were stained with REC8, which marks a cohesin subunit in meiotic cells and DDX4, which marks all germ cells. REC8 expression is evident only in 28 and 90 dpp in specific seminiferous tubules, indicated by white arrows. FIG. 6E shows images of whole mount immunostaining of REC8 and DDX4 on D7, D21 and D42 organoid cultured on transition media. White arrows indicate co-staining of REC8 and DDX4. Scale bars, 100 m;

FIG. 7—Sertoli cell-specific reporter mouse lines used for testicular harvest. Shown are Bright-field (BF) and fluorescent images of testes from either E12.5-13.5 embryos or P6 pups harvested from Sox9-IRES-GFP or TESCO-CFP mice. Scale bars, 500 m;

FIGS. 8A-B—Testicular cells cultured in 2D with serum-based media. FIG. 8A shows BF and fluorescent images of testicular cells harvested from either Sox9-IRES-GFP or TESCO-CFP pups. Cells were cultured for 7 days on 0.2% gelatin-coated dishes in Ad-DMEM/F12 media supplemented with 2% FBS. Scale bars, 100 m. FIG. 8B shows bar graphs of quantitative PCR (qPCR) analysis of testicular cells from TESCO-CFP mice, cultured for 1-7 days in serum-based media. Data are presented as mean 2^{-ΔΔCt} values SEM normalized to the housekeeping gene Hprt. *P<0.05, **P<0.01, ***P<0.001, and ****P<0.0001, ns—not significant. N=3;

FIGS. 9A-B—Testicular cells cultured in 2D with testis-defined media. FIG. 9A shows BF and fluorescent images of testicular cells harvested from either Sox9-IRES-GFP or TESCO-CFP pups. Cells were cultured for 7 days on 0.2% gelatin-coated dishes in defined media containing Ad-DMEM/F12 media supplemented with testicular growth factors. Scale bars, 100 m. FIG. 9B shows bar graphs of qPCR analysis of testicular cells from TESCO-CFP mice, cultured for 1-7 days with defined media. Data are presented as mean 2^{-ΔΔCt} values±SEM normalized to the housekeeping gene Hprt. *P<0.05, **P<0.01, ***P<0.001, and ****P<0.0001, ns—not significant. N=5;

FIGS. 10A-B—Testicular cells cultured on Geltrex coating in defined media. FIG. 10A shows BF and fluorescent images of testicular cells harvested from TESCO-CFP pups and cultured on various Geltrex dilutions (1:100, 1:50 and 1:20) or 0.2% gelatin (gel) coated wells. Scale bars, 100 m. FIG. 10B shows bar graphs of qPCR analysis of testicular cells from TESCO-CFP mice, cultured for 1-7 days on defined media and either Geltrex or Gelatin coating. Data are presented as mean 2^{-ΔΔCt} values±SEM normalized to the housekeeping gene Hprt. *P<0.05, **P<0.01, ***P<0.001, and ****P<0.0001, ns—not significant. N=5;

FIGS. 11A-B—Testicular neonatal organoids cultured on transwell inserts. FIG. 11A shows representative bright field (BF) and fluorescent images of organoids derived from Sox9-IRES-GFP testicular cells and cultured for up to 21 days on transwell inserts; FIG. 11 shows representative BF and fluorescent images of organoids derived from TESCO-CFP testicular cells and cultured for up to 21 days on transwell inserts. Scale bars, 100 μm;

FIG. 12—Organoids generated from mature testes. FIG. 12 shows Representative bright field (BF) images of organoids generated from P90 primary testicular cells, cultured for up to 21 days on transwell inserts in defined media. Scale bar, 500 m;

FIGS. 13A-D—Refinement of media composition for immature and mature state of testicular organoids. FIG. 13A shows a schematic representation of tested media compositions (B27-based or KSR-based). The invariable additions are labelled in gray and the growth factors added to each media are labelled in black. (Scheme created using BioRender). FIG. 13B shows bar graphs of qPCR analysis of Sertoli cell markers in organoids cultured in B27-based media for 14 days, compared to P5, P20 and P90 in vivo testis. Data are presented as mean 2^{-ΔΔCt} values±SEM normalized to the housekeeping gene Hprt. *P<0.05, **P<0.01, ***P<0.001, and ****P<0.0001, ns—not significant. N=4-5. FIG. 13C shows bar graphs of qPCR analysis of Sertoli cell markers in organoids cultured on KSR-based media for 14 days, compared to P5, P20 and P90 in vivo testis. Data are presented as mean 2^{-ΔΔCt} values±SEM normalized to the housekeeping gene Hprt. *P<0.05, **P<0.01, ***P<0.001, and ****P<0.0001, ns—not significant. N=4-5. FIG. 13D shows bar graphs of organoid area on days 7 and 14 in B27—and KSR-based media. D7 bars are in black, D14 bars are cumulative and shaded grey. Data are presented as mean area±SEM. Statistical significance is shown for mature 1 and mature 2 vs. immature media at day 7 of culture. *P<0.05, ns—not significant. N=5-8;

FIGS. 14A-D—Gene expression profiles of organoids cultured in transition media over time. FIGS. 14A-D show bar graphs of Quantitative RT-PCR performed on mRNA extracted from neonatal testes, or TESCO-CFP organoids cultured for the indicated number of days on transition media assessing Sertoli cell markers (FIG. 14A), Leydig cell markers (FIG. 14B) or other testicular cells markers (FIGS. 14C-14D). Gene names are indicated. D denotes culture day of organoids, grey. P denotes days postpartum (dpp) of in vivo testis, black. Data are presented as mean 2^{-ΔΔt} values±SEM normalized to the housekeeping gene Hprt. *P<0.05, **P<0.01, ***P<0.001, and ****P<0.0001, ns—not significant. N=3-6;

FIGS. 15A-B. Transition media promotes maturation of Sertoli and Leydig cells. FIGS. 15A-B show bar graphs of Quantitative RT-PCR, performed on mRNA extracted from neonatal testes, or TESCO-CFP organoids cultured for the indicated number of days on transition media assessing Sertoli cell immature vs. mature markers (FIG. 15A), or fetal Leydig cell vs. adult Leydig cell markers (FIG. 15B). Gene names indicated. D denotes culture day of organoids, grey. P denotes days postpartum (dpp) of in vivo testis, black. Data are presented as mean 2^{-ΔΔCt} values±SEM normalized to the housekeeping gene Hprt. *P<0.05, **P<0.01, ***P<0.001, and ****P<0.0001, ns—not significant. N=3-6;

FIGS. 16A-D—Testicular organoids on transition media preserve testicular structures and gonadal cell types for up to 9 weeks. FIGS. 16A-D show images of whole mount co-immunostaining of markers for the major types of testicular cells in organoids cultured for 7 days (FIG. 16A), 35 days (FIG. 16B), 49 days (FIG. 16C) and 63 days (FIG. 16D). SOX9 marks Sertoli cells, DDX4 labels gonocytes, 3BHSD marks Leydig cells and α-SMA labels PM cells. Images on the right are merged views of all channels. Scale bars, 100 m.

### DETAILED DESCRIPTION OF THE INVENTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

In the description and claims of the application, the words “contain”, “comprise”, “include” and “have”, and forms thereof, are not limited to members in a list with which the words may be associated.

The term “about” when referring to a measurable value such as an amount, a ratio, and the like, is meant to encompass variations of 10% of the indicated value, as such variations are also suitable to perform the disclosed invention. Any numerical values appearing in the application are intended to be construed as if preceded by “about”, unless indicated otherwise.

The term “protocol” as used herein is interchangeable with the term “method” or “process” in the context of a scheme for obtaining testicular organoids.

A “media” or “culture media,” as used herein, refers to an aqueous based solution, including one or more factors, that is provided for the maintenance, growth, viability, differentiation, maturation and/or storage of the organoids disclosed herein.

In some embodiments, as detailed herein, the culture media is a serum free media (i. e. The media does not include serum). In some embodiments, the culture media includes B-27 (also referred to as B27) supplement. B-27 Supplement is a serum-free supplement for cell culture, that may be used with basic mediums, such as DMEM/F12 or equivalents, to support cell viability and function. Components of B27 Supplement may include one or more of. Antioxidants, Vitamins (such as Vitamin A (retinyl acetate) and Vitamin E (α-tocopherol)), Minerals and Trace Elements, Hormones (such as transferrin and insulin), Fatty Acids and Lipids (such as linoleic acid and cholesterol) and optionally Growth Factors.

The term “Leydig cells” as used herein, refer to cells in the mammalian testis primarily responsible for producing androgens, such as testosterone. The cells include two key steroidogenic enzyme pathways, namely, cytochrome P450 side chain cleavage (P450scc) and 30-HSD. Leydig cells carry out the conversion of cholesterol, the substrate for all steroid hormones, to pregnenolone; and the conversion of pregnenolone to progesterone. The cells are naturally found in the interstitial tissue of the testes, situated between the seminiferous tubules where sperm production occurs. The cells are large, polygonal with an abundant smooth endoplasmic reticulum, lipid droplets, and a prominent nucleus, reflecting their role in steroid hormone synthesis. Leydig cells can be in an immature state (also referred to herein as Fetal Leydig cells (FLC) present in an immature testis) and these are replaced in adult testis with Adult Leydig cells (ALC).

The term “Sertoli cells” as used herein refer to specialized somatic cells found within the seminiferous tubules of mammalian testis. They play an essential role in supporting and regulating spermatogenesis (the production and maturation of sperm) and maintaining the testicular microenvironment. The cells are found inside the seminiferous tubules, surrounding and supporting developing germ cells (spermatogonia, spermatocytes, spermatids, and spermatozoa). The Sertoli cells are elongated, with extensive cytoplasmic processes that create a supportive network around germ cells. The cells provide both structural and nutritional support to germ cells as well as create a specialized blood barrier by forming tight junctions, separating the basal and adluminal compartments of the seminiferous tubules. Sertoli cells can be in an immature state (in immature testis) and a mature state (in mature or adult testis). Each of the states may be characterized by expression of cellular markers, as detailed herein below.

The term “germ cells” (“gonocytes”) relates to specialized cells that give rise to gametes (sperm) through the process of gametogenesis. Gonocytes are an early developmental stage of male germ cells found in the testes during fetal and neonatal life. Gonocytes are primordial germ cells (PGCs) that have migrated to the gonadal ridge during fetal development and have differentiated into male germ cells. The gonocytes are found in the seminiferous tubules of the fetal and neonatal testes and reside near the basement membrane during the early stages of development. Gonocytes are precursors to spermatogonial stem cells (SSCs), which will later give rise to spermatozoa. The gonocytes may express specific molecular markers, such as OCT4, PLZF, SOX2, KIT, and NANOG.

The term “Peritubular Myoid Cells” (“PM”) relates to specialized contractile cells located in the walls of the seminiferous tubules of the testes. These cells play a key role in maintaining the structural integrity of the tubules, facilitating sperm transport, and supporting spermatogenesis. The cells are found surrounding the seminiferous tubules, forming part of the tubular wall along with the basement membrane. The cells are flattened, spindle-shaped cells with smooth muscle-like features, including actin and myosin filaments for contractility. The cells express markers such as α-smooth muscle actin (α-SMA), desmin, and extracellular matrix proteins like collagen and laminin.

As used herein, the term “transwell” (also termed herein “Transwell” and “trans well”) relates to an insert as known in the art, which is configured to be positioned in a culture plate, or culture chamber, to allow cells to grow on a membrane of the insert, while providing a gas-liquid interphase to the cells, with media from the bottom and air from the top. When using a transwell insert, an upper chamber may be defined by the insert and include the cultured cells (and optionally additional substances, cells, etc.), and the lower chamber (well of the culture plate) can contain different culture media and optionally additional substances, cells, etc. In some examples, a transwell insert may include a membrane made of materials such as polycarbonate or polyester, with pore sizes typically ranging from 0.4 μm to 8 μm

According to some embodiments, there are provided herein advantageous methods for generating testes (testicular) organoids, which are viable and function in-vitro, over an extended period of time, while preserving spatial and/or temporal gene expression of one or more cellular markers and/or having structures which are comparable to those of a corresponding in-vivo testis.

According to some embodiments, in order to generate testicular organoids, transwell inserts, which provide a gas-liquid interphase, may be used for generating the testicular organoids disclosed herein.

According to some embodiments, various culture and media conditions are used to generate testicular organoids from primary testicular cells.

According to some embodiments, as exemplified herein, there are provided herein testis organoids that are formed from primary testicular cells of neonatal pups or embryos, and can be cultured in vitro for up to 9 weeks, while maintaining structure and gene expression profiles that closely resemble the in vivo testis at corresponding stages. These organoids preserve the presence of all major somatic cell types and germ cells, and organize into tubule-like structures and interstitial area.

According to some embodiments, there are further provided herein various culture media compositions that can promote the immature or mature cell states of Sertoli cells and Leydig cells, and hence provide a platform for in vitro maturation of the organoids.

According to some embodiments, the mature organoids disclosed herein can support entry of SSC to meiosis.

According to some embodiments, the herein disclosed testicular organoid system can provide a scalable approach for generating testicular models from neonatal or embryonal cells for use in various applications, such as, drug screening, elucidating disease conditions, and the like.

According to some embodiments, the disclosed methods/protocols are composed of a series of steps, where each step attempts to efficiently mimic/simulate in-vivo signaling environment sensed by the testicular cells at the relevant developmental stage, e.g. immature stage and mature stage.

According to some embodiments, the testis organoid disclosed herein can at least partially mimic the structural, physiological and/or functional properties of in vivo testicular tissue, including hormone production, germ cell support, and spermatogenesis.

According to some embodiments, as demonstrated herein below, the organoids can maintain viability for up to 9 weeks, during which they underwent growth and differentiation. In some embodiments, Sertoli cells and Leydig cells of the organoid demonstrate maturation trends consistent with in vivo testis development. In further embodiments, spermatogonial stem cells (SSCs) of the organoid can enter meiosis, indicating functionality for spermatogenesis.

According to some embodiments, the organoids disclosed herein can be used as a model for studying testis development and disorders, such as male infertility and disorders of sex development (DSDs), testing pharmacological effects, environmental toxins, and hormone interactions, and infertility.

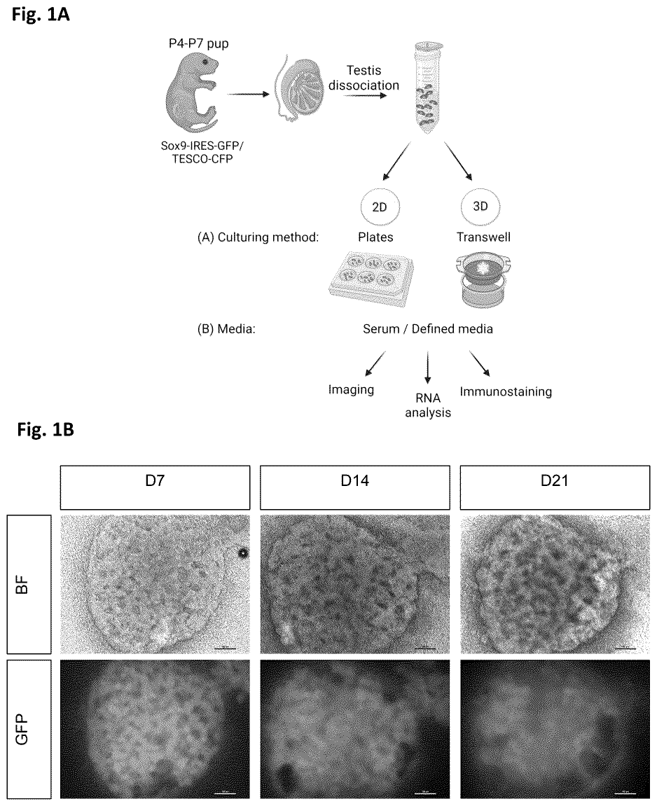

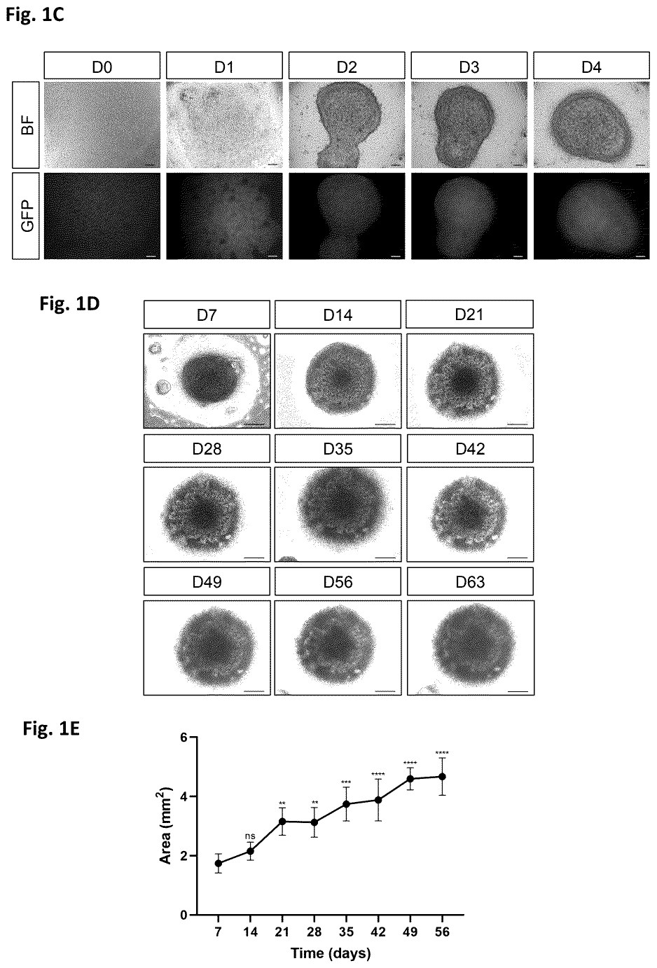

Reference is now made to FIG. 1A, which is a schematic illustrations of methods for generating testis organoids, according to some embodiments. As shown in FIG. 1A, testis are obtained from a neonatal murine pup (for example, at days P4-P7) and dissociated (by mechanical and/or enzymatic means) to obtain testis cells (including, for example, Sertoli cells, Leydig cells, PM cells and gonocytes). The cells are then plated on a Transwell insert, in the presence of a culture medium, and incubated for a period of time, to obtains 3D organoids. The obtained organoids may be analyzed by various methods (such as imaging, gene expression (RNA analysis), immunostaining) to determine various traits of the organoids, including, spatial and/or temporal expression of cellular markers, spatial and/or temporal structural changes, and the like. Likewise, for the production of organoids according to the method, testis cells obtained from embryonal testis can be used, as detailed herein below. As shown in FIG. 1A, the cells may be grown in 2D culture plates, however, as demonstrated herein below, surprisingly, when grown in culture plates, the testicular cells do not form an organoid.

According to some embodiments, there is thus provided a method for generating a testis organoid, the method includes one or more of the steps of:
a) Obtaining primary testis cells from a neonatal and/or embryonic testis;
b) Seeding/plating/culturing the obtained testis cells on a Transwell insert;
c) Maintaining/culturing the cells in the presence of a defined culture media, at suitable conditions (such as, temperature and humidity).
d) Optionally, the method may further include testing spatial and/or temporal expression patterns of one or more cellular markers and/or testing morphology of the organoids, at various stages of development thereof.

According to some embodiments, the testis may be murine testis or human testis.

In some embodiments, the testis may be obtained from neonatal animal (such as a mice). In some embodiments the testis may be obtained from a P2-P10 days old mice. In some embodiments, the testis may be obtained from P4-P7 days old mice.

In some embodiments, the testis may be obtained from embryos. In some embodiments the testis may be obtained at embryonic day E10-E16 of mice embryo. In some embodiments, the testis may be obtained at embryonic day E112.5-E14.5 of mice embryo. Embryos are collected after timed matings, with noon of the day of the vaginal plug is designated as E0.5.

According to some embodiments, after the testis is recovered, it may undergo mechanical and/or enzymatic dissociation. For example, the testis may be mechanically dissociated by dissecting (for example, by cutting into small pieces with forceps). Next, to obtain single cell testicular cell suspension, the obtained testicular pieces may be enzymatically treated (for example, using enzymes, such as, trypsin, collagenase, and the like.) According to some embodiments, for each organoid, at least about 1×10⁴-1×10⁷ cells may be plated/seeded. In some embodiments about 1×10⁵-1×10⁶ cells per organoid may be plated. In some embodiments, about 2-4×10⁵ cells per organoid may be plated. In some embodiments, about 2-2.5×10⁵ neonatal testis cells may be plated per organoid. In some embodiments, about 3-3.5×10⁵ embryonal testis cells may be plated per organoid. In some embodiments, each transwell insert may be plated with one or more organoids.

According to some embodiments, the size (area) of the organoid may range in the size of about 2-30 mm². In some embodiments, the size of the organoid may increase over time in culture. In some embodiments, for example, the size of the organoid may change from about 2 mm² at day P7 to about 4.5 mm² at day P56.

According to some embodiments, the plated cells may then be incubated under suitable conditions (such as, temperature in the range of 34-37 °C). In some embodiments, the organoids may be plated in the presence of a defined culture media, which facilitates the viability and functionality of the organoids.

According to some embodiments, a defined culture media is a serum free media. In some embodiments, a defined culture media may include components such as: Ad-DMEM-F12, Penicillin-Streptomycin, L-glutamine, B-27 supplement, N-Acetyl-L-cysteine, EGF, FSH, FGF9, Prostaglandin D2 (PDG2), Testosterone, Activin A, or any combinations thereof. Each possibility is a separate embodiment.

According to some embodiments, the defined media is based on DMED-F12 medium including B-27 supplement and one or more of. N-Acetyl-L-cysteine, Epidermal Growth Factor (EGF), Follicular stimulating hormone (FSH), Fibroblast Growth Factor (FGF), Prostaglandin D2 (PDG2), Testosterone and/or Activin A. Each possibility is a separate embodiment.

According to some embodiments, each of the EGF, FSH, FGF, PDG2, Testosterone and/or Activin A may be from various sources, including human, murine (rat or mice) and may a recombinant product, or an isolated product.

According to some embodiments, EGF may be added at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 40-55 ng/mL. Each possibility is a separate embodiment. In some embodiments, the concentration of EGF is about 50 ng/mL.

According to some embodiments, FSH may be added at a concentration of about 1-150 ng/ml, about 2 to about 100 ng/mL, about 5 to about 100 ng/mL, about 10 to about 80 ng/mL, about 15 to about 60 ng/mL, or about 15-25 ng/mL. Each possibility is a separate embodiment. In some embodiments, the concentration of FSH is about 22 ng/mL.

According to some embodiments, FGF (such as, FGF9) may be added at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 45-55 ng/mL. Each possibility is a separate embodiment. In some embodiments, the concentration of FGF is about 50 ng/mL.

According to some embodiments, PDG2 may be added at a concentration of about 0.1-1p g/ml, about 0.2 to about 0.9 μg/mL, about 0.3 to about 0.8 μg/mL, about 0.4 to about 0.6 μg/mL, or about 0.45-0.55 μg/mL. Each possibility is a separate embodiment. In some embodiments, the concentration of PDG2 is about 0.5 μg/ml.

According to some embodiments, testosterone may be added at a concentration of about 0.1-10 μM, about 0.2 to about 9 μM, about 0.3 to about 8 μM, about 0.4 to about 5 μM, or about 0.5 to about 3 μM, or about 0.8-1.2 μM. Each possibility is a separate embodiment. In some embodiments, the concentration of testosterone is about 1 μM.

According to some embodiments, Activin may be added at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 45-55 ng/mL. Each possibility is a separate embodiment. In some embodiments, the concentration of FGF is about 50 ng/mL.

According to some embodiments, B-27 supplement may be added at a concentration of 0.5×-5×, for example, 1×.

According to some embodiments, organoids may form even after 2 days in culture. In some embodiments, the organoids may be viable and/or functional for at least 2 weeks (14 days), at least 3 weeks (21 days), at least 4 weeks, at least 5 weeks, at least 6 weeks, at least 7 weeks, at least 8 weeks, at least 9 weeks. Each possibility is a separate embodiment.

According to some embodiments, surprisingly, as demonstrated herein below, the in-vitro organoids are capable or recapitulate gene expression profile (temporal and/or spatial) of various testis cellular markers. The cellular markers may be cell specific and state specific (for example, expressed according to the differentiation state of the cells).

According to some embodiments, cellular markers of Sertoli cells may include one or more of the following markers: Amh, Ar, Clu, Cx43, Cyp26b1, Dhh, Erbb4, Fgf9, Fshr, Gata4, Gdnf, Nr5a1, Ptgds, Shbg, Sox8, Sox9, Wt1, or any combinations thereof. Each possibility is a separate embodiment.

According to some embodiments, cellular markers of immature Sertoli cells may include, for example, Amh, Gata4, Gdnf, or any combinations thereof. Each possibility is a separate embodiment. According to some embodiments, cellular markers of mature Sertoli cells may include, for example, Cx43, Gata1, or any combinations thereof. Each possibility is a separate embodiment.

According to some embodiments, cellular markers of Leydig cells may include one or more of the following markers: 3βHsd, Crhr1, Cyp11a1, Cyp26b1, Gsg11, Hsd3b6, Ins13, Nr5a1, Star, Wt1, or any combinations thereof. Each possibility is a separate embodiment.

According to some embodiments, cellular markers of fetal Leydig cells may include, for example, Crhr1, Cyp26b1, Gsg11, or any combinations thereof. Each possibility is a separate embodiment. According to some embodiments, cellular markers of adult Leydig cells may include, for example, Hsd3b6.

According to some embodiments, cellular markers of SSC cells may include Plzf.

According to some embodiments, cellular markers of Peritubular myoid cells (PMC) may include αSma.

According to some embodiments, cellular markers of mature sperm may include Acrosin.

According to some embodiments, cellular markers of Meiotic spermatids may include Rec8 and Scyp3.

According to some embodiments, the methods disclosed here may further include maintaining immature state of the organoid, maintaining mature state of the organoid and/or promoting maturation of the organoid in-vitro. To this aim, use of a first culture media (also referred to herein as immature media) and/or of a second culture media (also referred to herein as mature media) may be used.

According to some embodiments, an immature culture media is a serum free media. In some embodiments, the immature culture media may include components such as: Ad-DMEM-F12, Penicillin-Streptomycin, L-glutamine, B-27 supplement, N-Acetyl-L-cysteine, EGF, FSH, FGF9, Activin A, IGF-1, or any combinations thereof. Each possibility is a separate embodiment.

According to some embodiments, the immature culture media is based on DMEM-F12 medium including B-27 supplement and one or more of: N-Acetyl-L-cysteine, Epidermal Growth Factor (EGF), Follicular stimulating hormone (FSH), Fibroblast Growth Factor (FGF), Insulin-like growth factor-1 (IGF-1) and/or Activin A. Each possibility is a separate embodiment.

According to some embodiments, EGF may be added to the immature culture media at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 40-55 ng/mL. Each possibility is a separate embodiment. In some embodiments, the concentration of EGF is about 50 ng/mL.

According to some embodiments, FSH may be added to the immature culture media at a concentration of about 1-150 ng/ml, about 2 to about 100 ng/mL, about 5 to about 100 ng/mL, about 10 to about 80 ng/mL, about 15 to about 60 ng/mL, or about 15-25 ng/mL. Each possibility is a separate embodiment. In some embodiments, the concentration of FSH is about 22 ng/mL.

According to some embodiments, FGF (such as, FGF9) may be added to the immature culture media at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 45-55 ng/mL. Each possibility is a separate embodiment. In some embodiments, the concentration of FGF is about 50 ng/mL.

According to some embodiments, IGF-1 may be added to the immature culture media at a concentration of about 1-300 ng/ml, about 5 to about 200 ng/mL, about 10 to about 150 ng/mL, about 10 to about 120 ng/mL, about 20 to about 110 ng/mL, or about 95-105 ng/mL. Each possibility is a separate embodiment. In some embodiments, the concentration of IGF-1 is about 100 ng/mL.

According to some embodiments, Activin may be added to the immature culture media at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 45-55 ng/mL. Each possibility is a separate embodiment. In some embodiments, the concentration of Activin is about 50 ng/mL.

According to some embodiments, B-27 supplement may be added to the immature culture media at a concentration of 0.5×-5×, for example, 1×.

According to some embodiments, a mature culture media is a serum free media. In some embodiments, the mature culture media may include components such as: Ad-DMEM-F12, Penicillin-Streptomycin, L-glutamine, B-27 supplement, N-Acetyl-L-cysteine, EGF, FSH, Testosterone, Thyroid hormone (T3), Retinoic acid (RA), Activin A, or any combinations thereof. Each possibility is a separate embodiment.

According to some embodiments, the immature culture media is based on DMEM-F12 medium including B-27 supplement and one or more of: N-Acetyl-L-cysteine, EGF, FSH, Testosterone, Thyroid hormone (T3), Retinoic acid (RA), and/or Activin A. Each possibility is a separate embodiment.

According to some embodiments, EGF may be added to the immature culture media at a concentration of about 1-200 ng/ml, about 2 to about 100 ng/mL, about 10 to about 100 ng/mL, about 10 to about 90 ng/mL, about 20 to about 70 ng/mL, about 30 ng/mL to about 60 ng/mL, or about 40-55 ng/mL. Each possibility is a separate embodiment. In some embodiments, the concentration of EGF is about 50 ng/mL.

According to some embodiments, FSH may be added to the mature culture media at a concentration of about 1-150 ng/ml, about 2 to about 100 ng/mL, about 5 to about 100 ng/mL, about 10 to about 80 ng/mL, about 15 to about 60 ng/mL, or about 15-25 ng/mL. Each possibility is a separate embodiment. In some embodiments, the concentration of FSH is about 22 ng/mL.

According to some embodiments, testosterone may be added at a concentration of about 0.1-10 μM, about 0.2 to about 9 μM, about 0.3 to about 8 μM, about 0.4 to about 5 μM, or about 0.5 to about 3 μM, or about 0.8-1.2 μM. Each possibility is a separate embodiment. In some embodiments, the concentration of testosterone is about 1 μM.

According to some embodiments, Thyroid hormone (T3) may be added to the mature culture media at a concentration of about 1-300 ng/ml, about 5 to about 200 ng/mL, about 10 to about 150 ng/mL, about 10 to about 120 ng/mL, about 20 to about 110 ng/mL, or about 95-105 ng/mL. Each possibility is a separate embodiment. In some embodiments, the concentration of Thyroid hormone (T3) is about 100 ng/mL.

According to some embodiments, Retinoic acid may be added at a concentration of about 0.1-10 μM, about 0.2 to about 9 μM, about 0.3 to about 8 μM, about 0.4 to about 5 μM, or about 0.5 to about 3 μM, or about 0.8-1.2 μM. Each possibility is a separate embodiment. In some embodiments, the concentration of Retinoic acid is about 1 μM.

According to some embodiments, Activin may be added to the mature culture media at a concentration of about 0.01-200 ng/ml, about 0.1 to about 100 ng/mL, about 0.1 to about 50 ng/mL, about 0.2 to about 50 ng/mL, about 0.5 to about 20 ng/mL, about 1 ng/mL to about 10 ng/mL, or about 3-8 ng/mL. Each possibility is a separate embodiment. In some embodiments, the concentration of Activin is about 5 ng/mL.

According to some embodiments, a method for promoting in-vitro maturation of organoids may include the use of the first culture media, to incubate the obtained testis cells (to promote proliferation thereof), and a after a period of time, replacing the first culture media with the second culture media and incubating the organoids (to promote differentiation/maturation thereof).

In some embodiments, a first step of such a transition protocol includes growing the primary neonatal or embryonic testis cells (obtained as detailed herein), under a first culture medium (immature medium) for a first period of time, and allowing maturation of the cells, under a second culture medium (mature medium), to facilitate the formation of mature organoids, having traits comparable to an in-vivo testis, for example, with respect of spatial and/or temporal expression of cellular markers, morphology of cells, cellular organization, biological activity, and the like, or any combinations thereof.

According to some embodiments, the methods disclosed herein allow maintaining the primary cells in an immature state and/or a mature state, based on the growth conditions in which the cells reside.

According to some embodiments, the first period of time may be in the range of about 2-21 days (from seeding), for example, 2-14 days, for example, 3-10 days, for example, about 10 days.

In some embodiments, the transition protocols combines features of immature and mature media to mimic the natural progression from immature to mature testicular states.

According to some embodiments, transgenic mouse strains that allow to track the presence and state of Sertoli cells may be used in the methods disclosed herein.

According to some embodiments, organoids in immature media may grow to a significantly larger size (area), due to the promotion of cell proliferation.

According to some embodiments, organoids in mature media may be smaller in size, reflecting reduced proliferation but higher differentiation.

According to some embodiments, the organoids may be tested to determine one or more properties thereof, including, for example, size, form, structure, function, gene expression patterns/profiles and the like.

According to some embodiments, various methodologies may be used to determine features of the organoids, including, for example, imaging (to determine size and/or structure), immunostaining (using one or more specific antibodies against molecular markers, such as, SOX9, AMH, CLD11 for Sertoli cells, DDX4 for germ cells, and 38HSD for Leydig cells), gene expression analysis (for example, by quantitative PCR (qPCR), to detect specific gene markers), and the like. According to some embodiments, the monitoring may be carried out using at least one technique selected from: high throughput two-photon 3D imaging, live imaging, immunostaining against marker proteins, RT-PCR, qPCR, FISH, and the like.

According to some embodiments, the methods may further include characterizing the cell populations in the 3D-organoids at different time points.

According to some embodiments, the methods may further include characterizing the spatial organization of the cell populations in the 3D-organoids at different time points.

According to some embodiments, as exemplified herein, the expression pattern of various cellular markers in the organoids may be similar to/resemble to the expression pattern of these markers in an in-vivo testis. For example, immature media can enhance expression of markers like Amh, Gata4, and Cx43, indicating a proliferative state of Sertoli cells, whereas, mature media enhance expression of markers, such as, Gata1, which are indicative of a mature Sertoli cell state.

According to some embodiments, as exemplified herein Leydig cells also undergo maturation as testis mature. While immature testis contains Fetal Leydig cells (FLC), these are replaced in adult testis with Adult Leydig cells (ALC). Among the FLC-specific genes are the Gsg11 and Crhr1 and an ALC-specific gene is the Hsd3b6 gene. According to some embodiments FLC markers are markedly decreased in culture when in mature media), while a major increase is seen with the expression of Hsd36b, which marks ALC.

According to some embodiments, the ability to manipulate the environment via immature, mature, and a transition protocol provides a versatile platform to model various aspects of testicular development, pathology, and spermatogenesis in vitro.

According to some embodiments, any of the methods disclosed herein may further include the addition of other, non-testis cells, such as, endothelial cells, in order to maintain and promote the organoids formation, viability and/or survival.

According to some embodiments, there is provided a testis organoid which is made of primary testis cells that have been obtained from neonatal and/or embryonic testis, wherein the cells form a 3D structure on a transwell insert, in the presence of a defined culture media, a mature culture media and/or a immature culture media, wherein the organoid is viable for at least three weeks in culture and is capable of:
a) expressing one or more cellular markers at a spatial and/or temporal pattern similar to that of an in-vivo testis;
b) have a structure similar to that of a corresponding in-vivo testis;
c) express Acrosin;
d) allowing at least some spermatogonial stem cells (SSC) of the organoid to enter meiosis; and/or
e) capable of maintaining germ cell function.

According to some embodiments, the organoid may mimic structural and functional characteristics of a natural testis, including, for example, hormone production and initiation of spermatogenesis.

According to some embodiments, surprisingly, the testicular organoid can maintain proper expression of various Sertoli cell gene markers, as well as Leydig cells, PM and SCC cells, for the entire culture duration, to levels comparable with in vivo testes at corresponding stages. Moreover, the organoid system was able to capture, and mimic trends of expression changes observed for some genes in vivo.

According to some embodiments, various Sertoli-specific markers, including, for example, Sox9, Nr5a1, Wt1, Clu and Ar may display similar levels of gene expression in the organoid culture as that of the in vivo testis throughout an 8-week culture. Some Sertoli markers are known to vary in expression in neonatal testis versus adult testis. For example, Amh, Fshr, Gdnf, and Gata4, decrease in expression, while Ptgds is increased. Surprisingly, as demonstrated herein, the culturing of the organoids over an extended period of time (for example, 5-9 weeks), can mimic these in-vivo expression trends, presenting decreased gene expression of Amh, Fshr, Gdnf, and/or Gata4 and increased expression of Ptgds over time. Each possibility is a separate embodiment.

According to some embodiments, expression of Leydig cell markers, such as, for example, 3ßHsd, Star, Ins13 and/or Cyp11a1 exhibit expression levels that are comparable to the in vivo testis, over a period of time. Each possibility is a separate embodiment.

According to some embodiments, expression of PM cells markers, such as, α-Sma, exhibit expression levels that are comparable to the in vivo testis, over a period of time.

According to some embodiments, expression of gonocytes cellular markers, such as, Plzf and/or Scyp3, which are expressed in meiotic cells, indicated that they are also expressed in the cultured organoid, similar to their expression in in-vivo testis.

According to some embodiments, the organoids includes all major testicular cell types therewithin, over an extended period of time in culture, including, Sertoli cells, Leydig cells, PM cells and SCC cells. According to some embodiments, immunostaining of organoids with specific cellular markers may be facilitated. Such markers may include, for example, SOX9, AMH and/or CLD11 for Sertoli cells, 3BHSD for Leydig cells, DDX4 for gonocytes and α-SMA for PM cells.

According to some embodiments, the organoids may have a spatial structural organization that closely resembles the in vivo testis. According to some embodiments, Sertoli cells may form numerous tubular structures (as may be evident by localization of AMH and CLD11 expressing cells). According to some embodiments, DDX4 positive cells (marking gonocytes) may be located next to Sertoli cells, and within the tubules. According to some embodiments, 38HSD-positive Leydig cells may be located outside of SOX9-positive tubules, in an area that corresponds to the interstitial area. According to some embodiments, α-SMA-positive PM cells may be located close to Sertoli cells.

According to some embodiments, the in vitro testicular organoids form compartments and structures that closely resemble the structure of the in vivo testis, and preserve all major cell types for prolonged periods of time.

According to some embodiments, the organoids may support entry of spermatogonial stem cells into meiosis. According to some embodiments, organoids may express the meiotic marker γH2AX, which labels double strand breaks (DSB), and/or REC 8 (a subunit of the cohesin complex which function during meiosis) and DDX4, which marks all stages of SSC and spermatogonia. γH2AX foci are normally present in intermediate and type B spermatogonia and in preleptotene to zygotene spermatocytes. Type A spermatogonia and round spermatids also stain for γH2AX, but as homogeneous nuclear staining. According to some embodiments, as exemplified herein, at least some DDX4-positive cells may also be γH2AX-positive, indicating that cells may enter meiosis within the organoids in vitro.

According to some embodiments, Acrosin, a gene specifically expressed at the mature sperm head within the acrosome is expressed in the in-vitro organoid as it matures.

According to some embodiments the in vitro organoid can support entry of SSC into meiosis, where at least some may even complete spermatogenesis and form a sperm head containing acrosin.

According to some embodiments, there is provided a testicular organoid cell compositions, which includes testis cells obtained/isolated from neonatal or embryonal testis.

According to some embodiments, there is provided a three-dimensional testicular organoid which includes a self-organized structure containing seminiferous tubule-like formations and heterogeneous population of cells, including spermatogonial stem cells, Sertoli cells, Leydig cells, and peritubular myoid cells.

According to some embodiments, the testicular organoid is devoid of immortalized cells.

According to some embodiments, the testicular organoid does not include immortalized cells.

According to some embodiments, there is provided a method for screening a substance for pharmacological activity, the method includes providing an organoid as disclosed herein, and adding the tested substance to the organoids, at one or more time points, for one or more periods of time, to determine the effect of the substance on the structure and/or function of the organoid.

The term “essentially devoid of”, as used herein with reference to ingredients excluded from a culture medium, means either that the respective culture medium does not contain any of the excluded ingredients or that any of the excluded ingredients are present at a level low enough not to cause any of the effects expected from the excluded ingredients.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Materials and Methods

### Mice

All animals were maintained with appropriate husbandry according to Bar Ilan University ethics protocols 57-08-2019 and 61-11-2020. Mouse strains TESCO-CFP [24] and Sox9-IRES-GFP [25] were maintained on an F1 (C57BL/6J X CBA) genetic background. Primers used for genotyping are listed in Table 1 below. Embryos and animals used were either TESCO-CFP homozygotes or Sox9-IRES-GFP heterozygotes for GFP.

TABLE 1
Primers for genotyping mice

| Primer | | | Product |
|---|---|---|---|
| name | Description | Sequence 5′ to 3′ | size |
| CFP-F | CFP | CGTGACCACCCTGACCTGG (SEQ ID NO: 1) | 327 bp |
| CFP-R | CFP | GTGGCGGATCTTGAAGTTGG (SEQ ID NO: 2) | |
| GFP-F | GFP | TGCAGTGCTTCAGCCGCTAC (SEQ ID NO: 3) | 462 bp |
| GFP-R | GFP | CCAGCAGGACCATGTGATCG (SEQ ID NO: 4) | |

### Harvesting Testicular Cells

Testes from P4-7 pups or adult mice were dissected and first dissociated mechanically by cutting into small pieces with forceps. Next, to obtain single cell testicular cell suspension, testicular pieces were incubated with final concentrations of 0.045% Trypsin (Thermo Scientific, 25300062) and 0.25% Collagenase II (Worthington, LS004176) for 30 min at 37 °C with gentle shaking every 10 min. Then, 8 ml of DMEM/F12 (Thermo Scientific 12634-010) medium containing 1× penicillin-streptomycin solution (Thermo Scientific, 15140122) and 2 mM L-glutamine (Biological Industries, BI-03-020-1A) were added to the suspension and the sample was filtered through a 70 μm cell strainer (Lifegene, CSS0100705). After centrifugation at 300×g for 5 min at 4 °C, cell pellet was resuspended in 10 ml medium, and cell number was counted using Countess II (Invitrogen).

For embryonic organoids, embryos were collected after timed matings at embryonic day E12.5-14.5, with noon of the day of plug designated as E0.5. Testes were harvested, separated from the mesonephros, and enzymatically dissociated as mentioned above for 8 min at 37 °C. Then 3 ml of DMEM/F12 medium containing 1× penicillin-streptomycin and 2 mM L-glutamine were added, cells were centrifuged at 300×g and cell pellet was resuspended in 1 ml medium and cell number was counted.

### Culturing Testicular Cells in 2D

Prior to seeding, 6-well plates (Corning 3516) were coated either with 0.2% gelatin (Sigma-Aldrich G9391) for at least 15 min at room temperature, or with Geltrex (Thermo Scientific A1413201) diluted in cold DMEM/F12 media at three different dilutions (1:100, 1:50, 1:20) for 60 min at 37 °C followed by 30 min at room temperature. When using serum-based media, cells from 12 testis of one P4-7 pup were seeded per well of a 6-well plate (roughly 0.6×10⁶ cells). When using defined medium, cells from one testis were seeded per well of a 6-well plate (roughly 1.2×10⁶ cells). Different amounts of cells were seeded in the serum-based or defined media due to the different expansion rate of the cells under both conditions. Cells were cultured for up to 7 days at 34 °C in a 5% CO₂ incubator. Media was replaced every 3-4 days. For media compositions see Table 2, below.

### Organoids Generation and Culture

Testicular cells were plated on Transwell 0.4 m pore polyester membranes (Corning 3450/Greiner 657641) at 2-2.5×10⁵ cells per organoid for embryonic organoids, and 3.5×10⁵ cells per organoid for neonatal and adult organoids. 7-9 organoids were seeded as pellets on one transwell insert with gaps in between the organoids. Organoids were cultured with the specified media (Table 2) at 34 °C in a 5% CO₂ incubator. Media was changed every 3-4 days from below the transwell inserts. For transition experiments, organoids were cultured for 10 days in immature media (Table 2) and then media was replaced to mature 2 media (Table 2) for an additional 11 days with a duration of 21 days in total.

### Media Compositions

The Following Media Compositions were Used in Various Experiments:

TABLE 2
Media composition

| | | Final | | Cat. |
|---|---|---|---|---|
| Media | Component | concentration | Manufacturer | number |
| Serum | Ad-DMEM-F12 | / | Thermo Fisher | 12634010 |
| based | | | Scientific | |
| medium | Penicillin- | 1x | Thermo Fisher | 15140122 |
| | Streptomycin | | Scientific | |

| | | | | |
|---|---|---|---|---|
| L-glutamine | 2 | mM | Biological | 03-020 |

| | | | | | |
|---|---|---|---|---|---|
| | | | | industries | |
| | | Fetal Bovine Serum | 2% | Thermo Fisher | 10270106 |
| | | (FBS) | | Scientific | |
| B27- | Defined | Ad-DMEM-F12 | / | Thermo Fisher | 12634010 |
| based | medium | | | Scientific | |
| media | | Penicillin- | 1x | Thermo Fisher | 15140122 |
| | | Streptomycin | | Scientific | |

| L-glutamine | 2 | mM | Biological | 03-020 |
|---|---|---|---|---|
| | | | industries | |
| B-27 supplement | 1X | | Thermo Fisher | 17504044 |
| | | | Scientific | |
| N-Acetyl-L-cysteine | 1.25 | mM | Sigma | A9165 |
| Mouse EGF | 50 | ng/ml | Thermo Fisher | PMG8043 |
| | | | Scientific | |
| Recombinant human | 22 | ng/ml | R&D Systems | 5925-FS |
| FSH | | | | |
| Recombinant mouse | 50 | ng/ml | R&D Systems | 7399-F9 |
| FGF9 | | | | |
| Prostaglandin D2 | 0.5 | μg/ml | Cayman | 12010 |
| (PDG2) | | | | |
| Testosterone | 1 | μM | Sigma | T1500 |
| Recombinant | 50 | ng/ml | R&D Systems | 338- AC |

| | | | | |
|---|---|---|---|---|
| | Human/Mouse/Rat | | | |
| | Activin A | | | |
| Immature | Ad-DMEM-F12 | / | Thermo Fisher | 12634010 |
| | | | Scientific | |
| | Penicillin- | 1x | Thermo Fisher | 15140122 |
| | Streptomycin | | Scientific | |

| L-glutamine | 2 | mM | Biological | 03-020- |
|---|---|---|---|---|
| | | | industries | 1A |
| B-27 supplement | 1X | | Thermo Fisher | 17504044 |
| | | | Scientific | |
| N-Acetyl-L-cysteine | 1.25 | mM | Sigma | A9165 |
| Mouse EGF | 50 | ng/ml | Thermo Fisher | PMG8043 |
| | | | Scientific | |
| Recombinant human | 22 | ng/ml | R&D Systems | 5925-FS |
| FSH | | | | |
| Recombinant mouse | 50 | μg/ml | R&D Systems | 7399-F9 |
| FGF9 | | | | |
| Recombinant | 50 | ng/ml | R&D Systems | 338- AC |
| Human/Mouse/Rat | | | | |
| Activin A | | | | |
| Recombinant human | 100 | ng/ml | R&D Systems | 291-G1 |

| | | | | |
|---|---|---|---|---|
| | IGF-1 | | | |
| Mature1 | Ad-DMEM-F12 | / | Thermo Fisher | 12634010 |
| | | | Scientific | |
| | Penicillin- | 1x | Thermo Fisher | 15140122 |
| | Streptomycin | | Scientific | |

| L-glutamine | 2 | mM | Biological | 03-020- |
|---|---|---|---|---|
| | | | industries | 1A |
| B-27 supplement | 1X | | Thermo Fisher | 17504044 |
| | | | Scientific | |
| N-Acetyl-L-cysteine | 1.25 | mM | Sigma | A9165 |
| Mouse EGF | 50 | ng/ml | Thermo Fisher | PMG8043 |
| | | | Scientific | |
| Testosterone | 1 | μM | Sigma | T1500 |
| Thyroid hormone (T3) | 100 | nM | Tocris | 6666 |
| Retinoic acid | 1 | μM | Sigma | R2625 |

| | | | | |
|---|---|---|---|---|
| Mature2 | Ad-DMEM-F12 | / | Thermo Fisher | 12634010 |
| | | | Scientific | |
| | Penicillin- | 1x | Thermo Fisher | 15140122 |
| | Streptomycin | | Scientific | |

| L-glutamine | 2 | mM | Biological | 03-020- |
|---|---|---|---|---|
| | | | industries | 1A |
| B-27 supplement | 1X | | Thermo Fisher | 17504044 |
| | | | Scientific | |
| N-Acetyl-L-cysteine | 1.25 | mM | Sigma | A9165 |
| Mouse EGF | 50 | ng/ml | Thermo Fisher | PMG8043 |
| | | | Scientific | |
| Recombinant human | 22 | ng/ml | R&D Systems | 5925-FS |
| FSH | | | | |
| Testosterone | 1 | μM | Sigma | T1500 |
| Thyroid hormone (T3) | 100 | nM | Tocris | 6666 |
| Retinoic acid | 1 | μM | Sigma | R2625 |
| Recombinant | 5 | ng/ml | R&D Systems | 338- AC |

| | | | | | |
|---|---|---|---|---|---|
| | | Human/Mouse/Rat | | | |
| | | Activin A | | | |
| KSR- | KSR | Ad-DMEM-F12 | / | Thermo Fisher | 12634010 |
| based | | | | Scientific | |
| media | | Penicillin- | 1x | Thermo Fisher | 15140122 |
| | | Streptomycin | | Scientific | |

| L-glutamine | 2 | mM | Biological | 03-020- |
|---|---|---|---|---|
| | | | industries | 1A |
| KnockOut Serum | 10% | | Thermo Fisher | 10828028 |
| Replacement (KSR) | | | Scientific | |
| N-Acetyl-L-cysteine | 1.25 | mM | Sigma | A9165 |

| | | | | |
|---|---|---|---|---|
| KSR- | Ad-DMEM-F12 | / | Thermo Fisher | 12634010 |
| Defined | | | Scientific | |
| | Penicillin- | 1x | Thermo Fisher | 15140122 |
| | Streptomycin | | Scientific | |

| L-glutamine | 2 | mM | Biological | 03-020- |
|---|---|---|---|---|
| | | | industries | 1A |
| KnockOut Serum | 10% | | Thermo Fisher | 10828028 |
| Replacement (KSR) | | | Scientific | |
| N-Acetyl-L-cysteine | 1.25 | mM | Sigma | A9165 |
| Mouse EGF | 50 | ng/ml | Thermo Fisher | PMG8043 |
| | | | Scientific | |
| Recombinant human | 22 | ng/ml | R&D Systems | 5925-FS |
| FSH | | | | |
| Recombinant mouse | 50 | μg/ml | R&D Systems | 7399-F9 |
| FGF9 | | | | |
| Prostaglandin D2 | 0.5 | μg/ml | Cayman | 12010 |
| (PDG2) | | | | |
| Testosterone | 1 | μM | Sigma | T1500 |
| Recombinant | 50 | ng/ml | R&D Systems | 338- AC |

| | | | | |
|---|---|---|---|---|
| | Human/Mouse/Rat | | | |
| | Activin A | | | |
| Immature | Ad-DMEM-F12 | / | Thermo Fisher | 12634010 |
| | | | Scientific | |
| | Penicillin- | 1x | Thermo Fisher | 15140122 |
| | Streptomycin | | Scientific | |

| L-glutamine | 2 | mM | Biological | 03-020- |
|---|---|---|---|---|
| | | | industries | 1A |
| KnockOut Serum | 10% | | Thermo Fisher | 10828028 |
| Replacement (KSR) | | | Scientific | |
| N-Acetyl-L-cysteine | 1.25 | mM | Sigma | A9165 |
| Mouse EGF | 50 | ng/ml | Thermo Fisher | PMG8043 |
| | | | Scientific | |
| Recombinant human | 22 | ng/ml | R&D Systems | 5925-FS |
| FSH | | | | |
| Recombinant mouse | 50 | μg/ml | R&D Systems | 7399-F9 |
| FGF9 | | | | |
| Recombinant | 50 | ng/ml | R&D Systems | 338- AC |
| Human/Mouse/Rat | | | | |
| Activin A | | | | |
| Recombinant human | 100 | ng/ml | R&D Systems | 291-G1 |

| | | | | |
|---|---|---|---|---|
| | IGF-1 | | | |
| Mature1 | Ad-DMEM-F12 | / | Thermo Fisher | 12634010 |
| | | | Scientific | |
| | Penicillin- | 1x | Thermo Fisher | 15140122 |
| | Streptomycin | | Scientific | |

| L-glutamine | 2 | mM | Biological | 03-020- |
|---|---|---|---|---|
| | | | industries | 1A |
| KnockOut Serum | 10% | | Thermo Fisher | 10828028 |
| Replacement (KSR) | | | Scientific | |
| N-Acetyl-L-cysteine | 1.25 | mM | Sigma | A9165 |
| Mouse EGF | 50 | ng/ml | Thermo Fisher | PMG8043 |

| | | | |
|---|---|---|---|
| | | Scientific | |
| Testosterone | Sigma | T1500 | T1500 |
| Thyroid hormone (T3) | Tocris | 6666 | 6666 |
| Retinoic acid | Sigma | R2625 | R2625 |

RNA Isolation, cDNA Preparation and qRT-PCR

Total RNA from mouse testis or organoids was extracted using TRozo™ Reagent (Thermo Scientific, 15596026) according to the manufacturer's protocol. Normally, 3-4 organoids were used for an RNA sample. RNA yield was quantified using a NanoDrop spectrophotometer, and 1500 ng RNA was treated with RQ1 DNase (Promega M610A) and used to synthesize cDNA using SuperScript™ III Reverse Transcriptase (Thermo Scientific 18080085). qRT-PCR reactions were performed in duplicate using Power SYBR Green PCR Master Mix (Thermo Scientific 4367659) and 140 nM each of forward and reverse primers and analyzed on the QuantStudio 1 Real-Time PCR System (Thermo Scientific). Primers used are listed in Table 3, below.

TABLE 3
Primers for Real-time quantitative RT-PCR

| Primer | | | | |
|---|---|---|---|---|
| name | Gene | Marker of | Sequence 5′ to 3′ | SEQ ID NO: |
| m3ßHsd_F | 3βHsd | Leydig cells | CTCAGTTCTTAGGCTTCAGCA | 5 |
| | | | ATTAC | |
| m3ßHsd_R | | | CCAAAGGCAAGATATGATTT | 6 |
| | | | AGGA | |
| mAcrosin_F | Acrosin | Mature sperm | TGTCCGTGGTTGCCAAGGATA | 7 |
| | | | ACA | |
| mAcrosin_R | | | AATCCGGGTACCTGCTTGTGA | 8 |
| | | | GTT | |
| mAmh_F | Amh | Immature | CCACGGTTAGCACCAAATAG | 9 |
| | | | C | |
| mAmh_R | | Sertoli cells | CACACAGAACCTCTGCCCTAC | 10 |
| | | | TC | |
| mAr_F | Ar | Sertoli cells | CTGGGAAGGGTCTACCCAC | 11 |
| mAr_R | | | GGTGCTATGTTAGCGGCCTC | 12 |
| mClu_F | Clu | Sertoli cells | TGAAGGGCCAGTGTGAAAAG | 13 |
| | | | T | |
| mClu_R | | | TTGAACAGTCCACAGACAAG | 14 |
| | | | ATCTC | |
| mCrhr1_F | Crhr1 | Fetal Leydig | CCAGGATCAGCAGTGTGAGA | 15 |
| mCrhr1_R | | cells | TGTTTGTGGTGTTGTAGCGG | 16 |
| mCx43_F | Cx43 | Adult Sertoli cell | GGTCTGAGAGCCCGAACTCTC | 17 |
| | | | CT | |
| mCx43_R | | | CCCATGTCTGGGCACCTCTCT | 18 |
| | | | TT | |
| mCyp11a1_F | Cyp11a1 | Leydig cells | CACTTCTGGAGGGAGAGTGG | 19 |
| mCyp11a1_R | | | ATGCCTGGAAGAAAGACCGA | 20 |
| mCyp26b1_F | Cyp26 | Sertoli cells/ | AAGGGCTCCATGGGATTC | 21 |
| mCyp26b1_R | bl | FLC | ACGACTGGAAGCCGGAAC | 22 |
| mDhh_F | Dhh | Sertoli cells | CCGCAACCACATCCACGTA | 23 |
| mDhh_R | | | CGGACCGCCAGTGAGTTATC | 24 |
| mErbb4_F | Erbb4 | Sertoli cells | CAGCGCTTCTCAGTCAGTGT | 25 |
| mErbb4_R | | | CTGCTGTTCCAGGTCAGAGA | 26 |
| mFgf9_F | Fgf9 | Sertoli cells | TCTTCCCCAACGGTACTATCCA | 27 |
| mFgf9_R | | | CGAAGCGGCTGTGGTCTTT | 28 |
| mFshr_F | Fshr | Sertoli cells | TTGCCTGATGATGTTTTCCA | 29 |
| mFshr_R | | | CTGGCCCTCAACTTCTTCAG | 30 |
| mGata1_F | Gata1 | Adult Sertoli cell | TGGGGACCTCAGAACCCTTG | 31 |
| mGata1_R | | | GGCTGCATTTGGGGAAGTG | 32 |
| mGata4_F | Gata4 | Sertoli cells | CCCCAATCTCGATATGTTTGA | 33 |
| | | | TG | |
| mGata4_R | | | TTGACACACTCTCTGCCTTCT | 34 |
| | | | GA | |
| mGdnf_F | Gdnf | Sertoli cells | GGGTGCGTTTTAACTGCCATA | 35 |
| mGdnf_R | | | GCCCAAACCCAAGTCAGTGA | 36 |
| mGsg11_F | Gsg1l | Fetal Leydig | GAGGAGCCGACGTTCATAGA | 37 |
| mGsg11_R | | cells | AGCACTGACGGTTTAGCTCT | 38 |
| mHprt_F | Hprt | HK gene | GCAGTACAGCCCCAAAATGG | 39 |
| mHprt_R | | | GGTCCTTTTCACCAGCAAGCT | 40 |
| mHsd3b6_F | Hsd3b | Adult Leydig | GGAGGAGATCAGGGTCCTGG | 41 |
| mHsd3b6_R | 6 | cells | CTAGGATGGTCTGCCTGGG | 42 |
| mIns13_F | Insl3 | Leydig cells | GCTGCTACTGATGCTCCTGG | 43 |
| mIns13_R | | | CTAGACCCGCAGCTTCCTC | 44 |
| mNr5a1_F | Nr5a1 | Sertoli cells | CCTCGATGTGAAATTCCTGAA | 45 |
| | | | CA | |
| mNr5a1_R | | | TCCTGGGCGTCCTTTACG | 46 |
| mPlzf_F | Plzf | | CCAGTTGGAGACGACCTACA | 47 |
| mPlzf_R | | Spermatogonial | GTGGCAGAGTTTGCACTCAA | 48 |
| | | stem cells (SSC) | | |
| mPtgds_F | Ptgds | Sertoli cells | GGCTCCTGGACACTACACCT | 49 |
| mPtgds_R | | | CATAGTTGGCCTCCACCACT | 50 |
| mRec8_F | Rec8 | Meiotic | GCCCTAGAAGGTGCTGGTTTG | 51 |
| | | | G | |
| mRec8_R | | spermatids | GTGGGGTCACCTCAGTGAGT | 52 |
| | | | AGG | |
| mScyp3_F | Scyp3 | Meiotic | GCAGTCTAGAATTGTTCAGAG | 53 |
| | | | CCAGA | |
| mScyp3_R | | spermatids | TCCAAACTCTTTATGAACTGC | 54 |
| | | | TCGTG | |
| mShbg_F | Shbg | Sertoli cells | CCAAAATCAGCAAACCCCAT | 55 |
| | | | T | |
| mShbg_R | | | CTGGATCCCAGGTTCGAAAC | 56 |
| mSox8_F | Sox8 | Sertoli cells | AGCGAGAAGAGGCCGTTTG | 57 |
| mSox8_R | | | TCAGTACCAGAGTCTGAGTCG | 58 |
| mSox9_F | Sox9 | Sertoli cells | AAGAAAGACCACCCCGATTA | 59 |
| | | | CA | |
| mSox9_R | | | CAGCGCCTTGAAGATAGCATT | 60 |
| mStar_F | Star | Leydig cells | GACGTCGGAGCTCTCTGCTT | 61 |
| mStar_R | | | GCCTTCTGCATAGCCACCTC | 62 |
| mWt1_F | Wt1 | Sertoli cells | TTGAATGCATGACCTGGAATC | 63 |
| | | | A | |
| mWt1_R | | | TTCCCTTTAAGGTAGCTCCTA | 64 |
| | | | GGTT | |
| maSma_F | aSma | Peritubular | CCCCTGAAGAGCATCCGACA | 65 |
| | | myoid cells | TGGCGGGGACATTGAAGGT | 66 |
| maSma_R | | (PMC) | | |

### Imaging of Organoids and Measurement of Organoids Size

All bright field and fluorescent images of organoids were taken using the Nikon Eclipse Ts2R microscope. For organoids size assessment, bright field images were taken on a Nikon Eclipse Ts2R microscope, and the organoid area (N=3-14 organoids) was measured using NIS-Elements D software.

### Statistical Analysis

Statistical analyses were carried out using Prism 9 software (GraphPad). The analyses used were either one-way ANOVA followed by Dunnett's, Kruskal-Wallis followed by Dunn's or Brown-Forsythe and Welch followed by Dunnett's post hoc tests, depending on the dataset. When both media compositions and times were compared, two-way ANOVA followed by Tukey's/Dunnett's was used. All experiments were repeated at least three independent times.

### Frozen Sections and Whole Mount Immunostaining

Testes were harvested and fixed overnight in 4% PFA (Sigma P6148) at 4 °C while rotating. Then, testes were washed three times in PBS+0.1% Triton X-100 (PBST) (Sigma 9002-93-1) and incubated at 4 °C in 20% sucrose (Fisher BioReagents BP220-1) until embedded in OCT (Leica Biosystems 14020108926). Embedded samples were sectioned at 10 μm-thick sagittal sections using a cryostat (Leica Biosystems CM3050S). Antigen retrieval was performed with DAKO (Target retrieval solution, Agilent S1699) at 65 °C for 30 min. Samples were then blocked in PBST containing 10% donkey serum (Sigma Aldrich D9663) for 1 h and incubated with primary antibodies (diluted in PBST containing 1% donkey serum) overnight at 4 °C (All primary and secondary antibodies used are listed in Table 4). After three washes with PBST, secondary antibodies and 4′,6-diamidino-2-phenylindole (DAPI, Molecular Probes, Thermo Scientific D-1306) were added for 1 h at room temperature. Slides were then washed, dried, and mounted (Polysciences 18606).

For wholemount immunostainings, organoids were fixed in 4% PFA for 20 min at room temperature. Blocking was performed in 5% donkey serum in PBS+0.3% Triton X-100 solution for 2-3 h at room temperature. Primary antibodies were incubated in PBS+0.3% Triton X-100 solution supplemented with 10% donkey serum overnight at 4 °C. Following six PBS+0.3% Triton X-100 washes, organoids were incubated with secondary antibodies and DAPI overnight at 4 °C. They were then washed in PBS+0.3% Triton X-100 and transferred onto glass slides with a mounting solution. All primary and secondary antibodies used are listed in Table 4. Images were obtained with a Leica Microsystems SP8 confocal microscope.

TABLE 4
Antibodies

| Protein | Cat. No. | Raised in | Company | Dilution |
|---|---|---|---|---|
| SOX9 | AB5535 | Rabbit | Merck Millipore | 1:5000 |
| DDX4 | ab27591 | Mouse | Abcam | 1:300 |
| αSMA | A2547 | Mouse | Sigma | 1:200 |
| 3βHSD | sc-515120 | Mouse | Santa Cruz | 1:200 |
| γH2AX | ab11174 | Rabbit | Abcam | 1:1000 |
| TRA98 | ab82527 | Rat | Abcam | 1:200 |
| AMH | sc-6886 | Goat | Santa Cruz | 1:200 |
| CLAUDIN-11 | Zy-364500 | Rabbit | Invitrogen | 1:100 |
| REC8 | ab192241 | Rabbit | Abcam | 1:200 |

### Example 1: Culturing Primary Testicular Cells in 2D Using Serum-Based or Defined Media

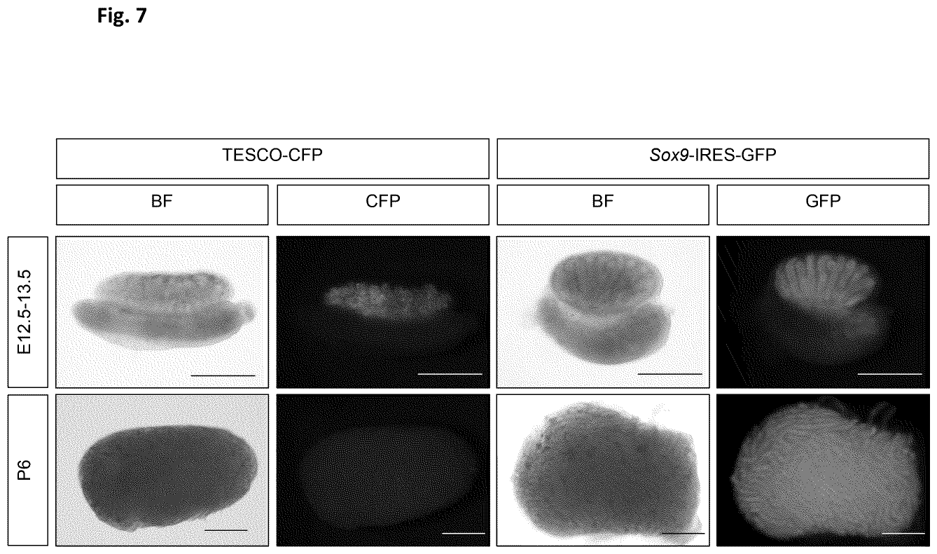

In order to determine optimal culture and media conditions for the generation of testicular organoids, two reporter mouse lines that harbor a fluorescent protein that specifically labels Sertoli cells were used. This enables tracking of the state of Sertoli cells, and expression levels of Sox9, one of the key markers of Sertoli cells, within the organoids over time. A first mouse line is the TESCO-CFP strain that contains the Sox9 testis-specific enhancer, TESCO, upstream of the CFP reporter [24]. The second mouse line is the Sox9-MRES-GFP strain that contains an IRES-GFP downstream of the Sox9 coding sequence [25]. Both reporter mouse lines express CFP or GFP within Sertoli cells of embryonic and adult gonads. Results are presented in FIG. 7.

For developing testicular organoids, calibrations were performed with postnatal day (P)4-7 neonatal testis. Entire testes from P4-P7 pups of the Sox9-IRES-GFP or TESCO-CFP mouse strains were harvested, dissociated into single cells, and plated on either 2D or 3D culture settings in various media compositions. The outcome of the various conditions was assessed by bright field (BF) and fluorescence examination of the cells or organoids, gene expression analysis, and immunostaining. The results are presented in FIG. 1A.

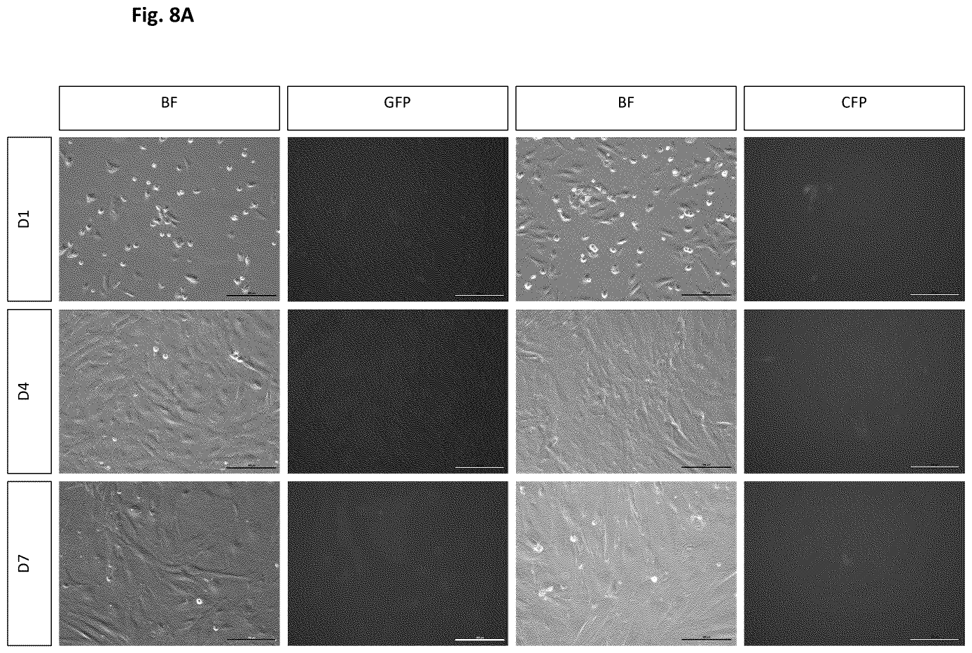

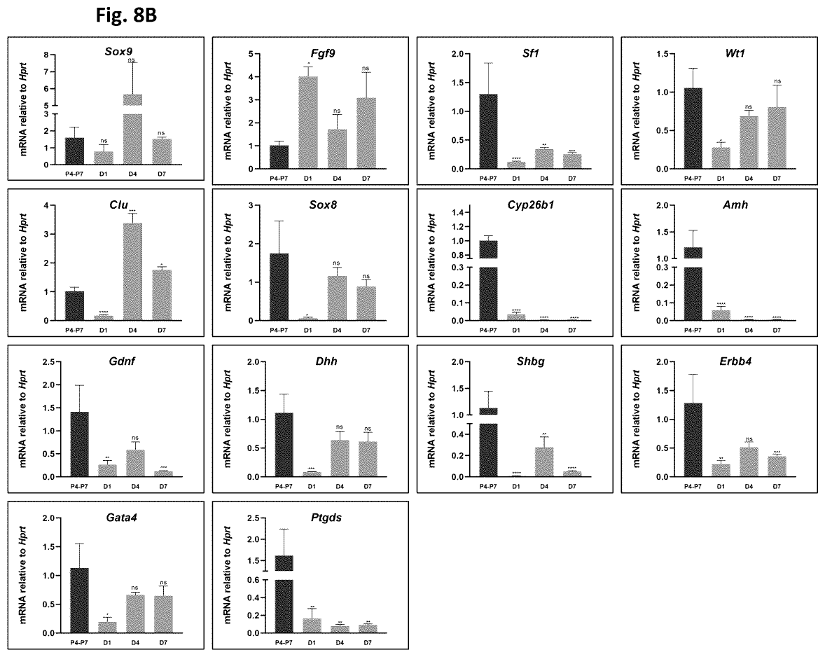

Previously described conditions for culturing primary testicular cells in standard 2D culture plates with media containing 2% serum [11] were used for baseline comparison. Plating primary testicular cells on plastic gelatin-coated dishes with Ad-DMEM/F12 media containing 2% fetal bovine serum (FBS) is not optimal for maintaining testicular cells, as can be deduced from the results presented in FIGS. 8A-B. Under such conditions, the cells adopted a fibroblast-like morphology, had a very dim expression of GFP or CFP and did not form aggregates, characteristic of Sertoli cells, as shown in FIG. 8A. Analyzing expression levels of several Sertoli cell markers at days (D)1, 4 and 7 of culture, compared to P4-P7 primary cells that were not cultured in vitro, indicated a drastic decrease in genes such as Amh, Cyp26b1, Ptgds and others. Expression of other genes was unstable during the culture period, and differed from the normal levels of expression in neonatal testis, as shown in FIG. 8B.

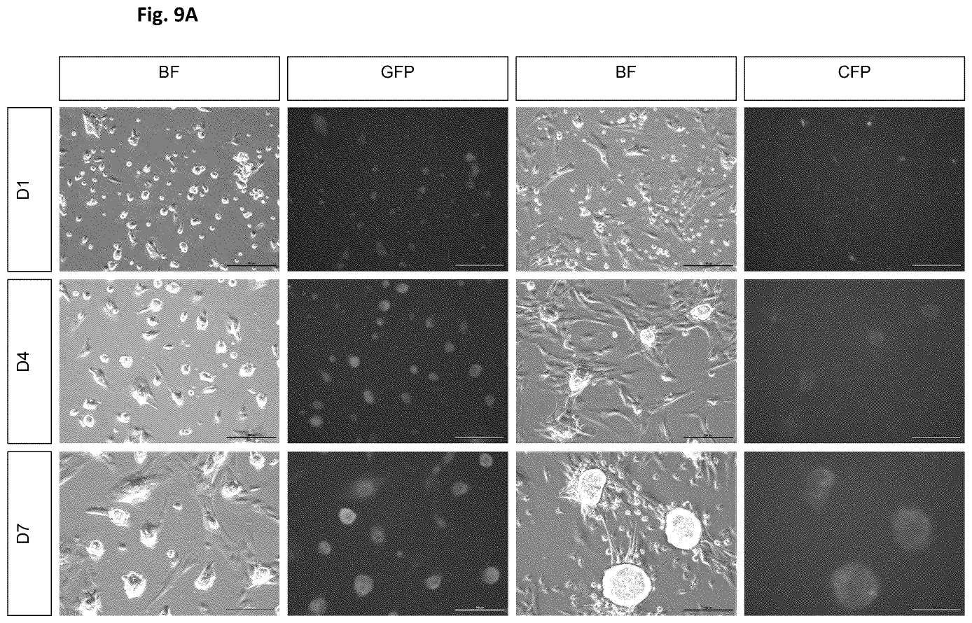

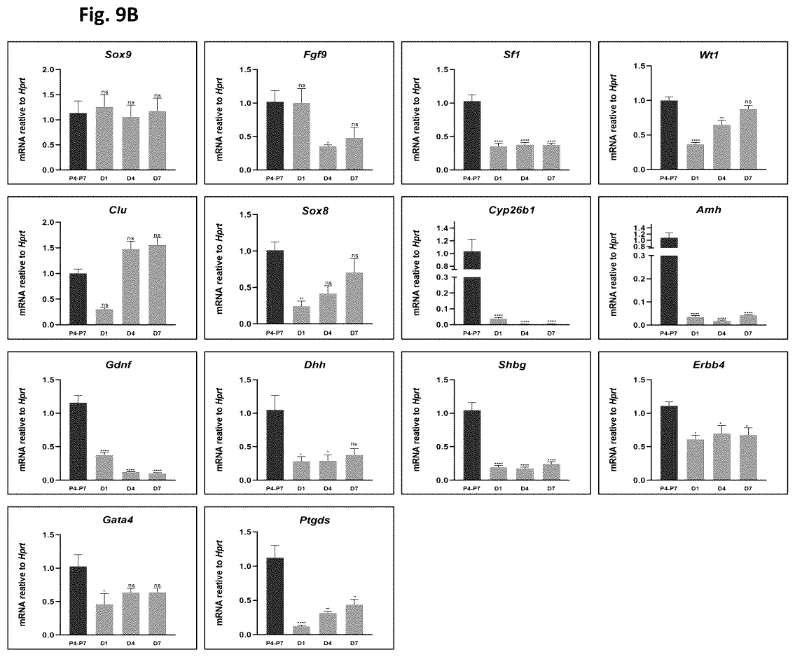

Next, it was sought to develop a “testis-defined media” (referred to herein “defined media”) containing factors that are normally present in the testis (as detailed in Table 2). The refined media contains recombinant human follicle-stimulating hormone (rhFSH) [26], fibroblast growth factor 9 (FGF9) [27], prostaglandin D2 (PGD2) [28, 29], testosterone [30] and activin A [27, 31]. Interestingly, culturing primary testicular cells on gelatin-coated dishes in defined media resulted in completely different cell morphology compared to the serum containing media used above, as shown in FIG. 9A. The cells formed rounded aggregates expressing CFP/GFP, and thus could be identified as Sertoli cells. Beneath the aggregates there were cells that attached to the plates, forming a “feeder-like layer” to which the aggregates attached (FIG. 9A). Despite the major morphological change in the cells, gene expression profiles of many Sertoli markers, such as, Amh, Cyp26b1, Dhh and Shbg, were still markedly decreased compared to those of primary cells. Other genes, such as Sox9, were more stable, as demonstrated in FIG. 9B. This indicates that media change alone, although influential, is not enough to enable proper prolonged culture of primary testicular cells.

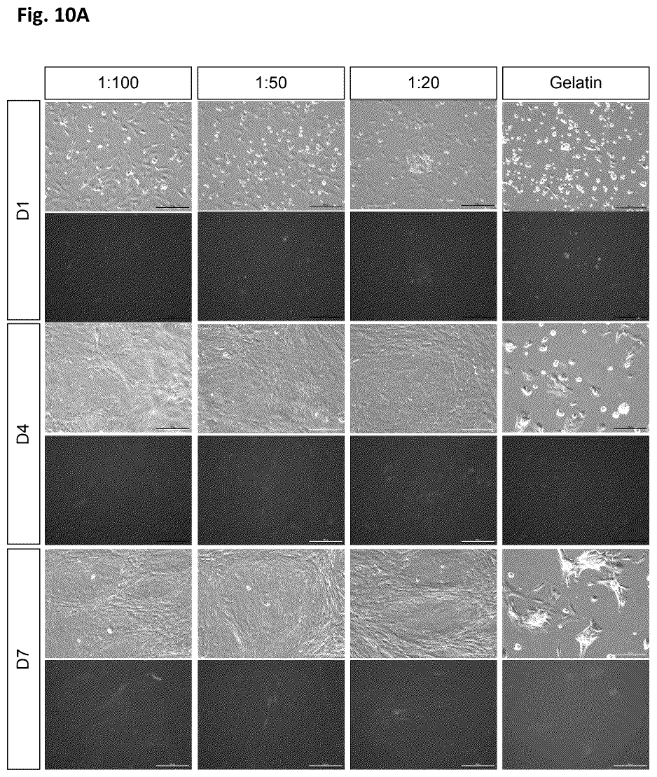

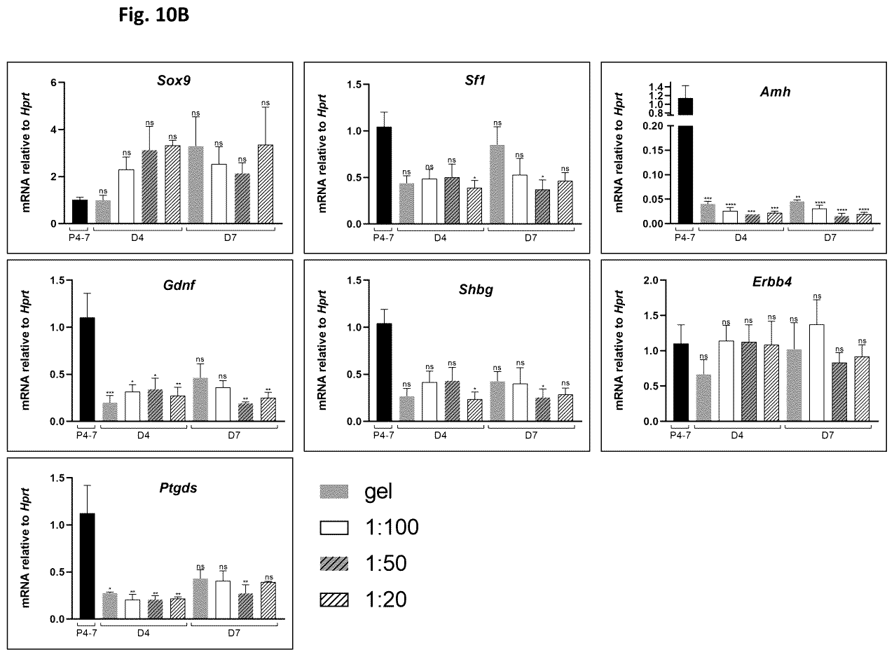

Next, primary testicular cells were seeded on top of various dilutions of Matrigel and were compared to cells grown in wells coated with gelatin. The results are presented in FIG. 10A. Bright field and fluorescent microscopy demonstrated that the cells were unable to form aggregates and presented very dim CFP expression. qPCR analysis demonstrated a low expression of many Sertoli markers, as shown in FIG. 10B. Thus, seeding on top of Matrigel could not support testicular cell growth in culture.

### Example 2: Culturing Primary Testicular Cells on Transwell Inserts with Defined Media

As culturing in various 2D culture conditions did not result in an optimal cell morphology, a 3D culture system including transwell insert was used. The experimental outline is shown in FIG. 1A.

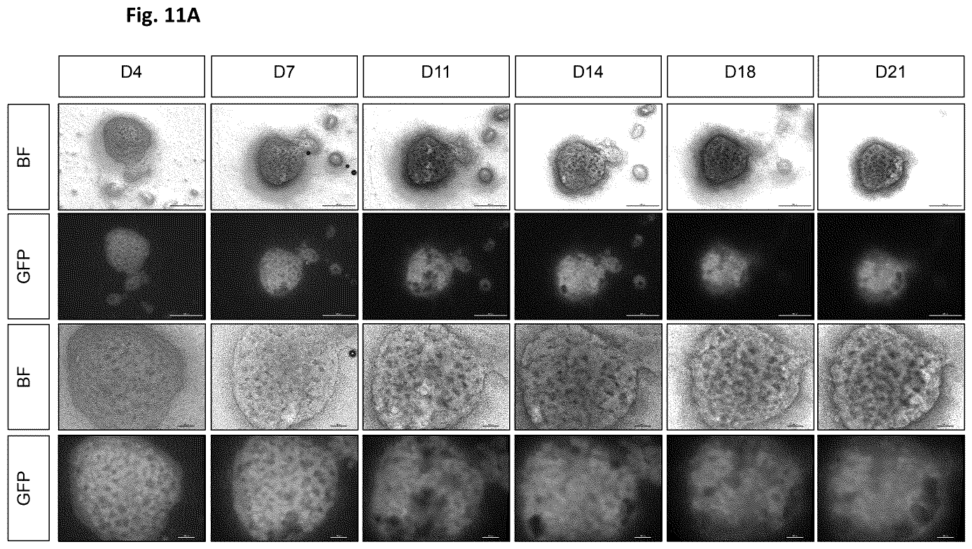

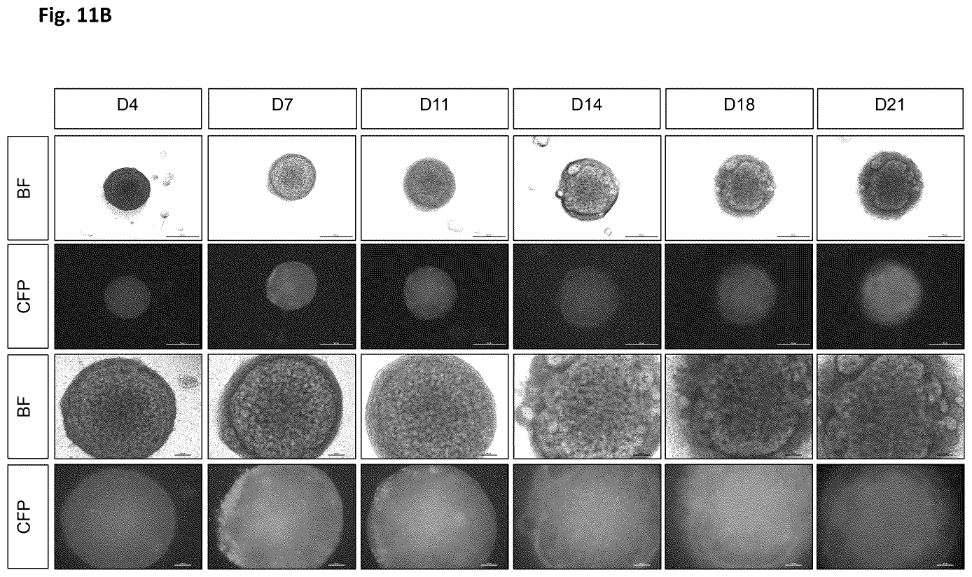

To this end, 3.5×10⁵ primary testicular cells per organoid were seeded on transwell inserts and defined media (Table 2) was added to the bottom of the culture well. When cultured on transwell inserts, the cells encounter a liquid-gas interphase with media from the bottom and air from the top. As shown in FIG. 1B and FIGS. 11A-B, testis organoids were formed, wherein the organoids presented clear tubular structures and very strong CFP or GFP expression that persisted for 21 days in culture.

To better assess the early stages of organoid formation, organoids were seeded and examined immediately after seeding and for 4 consecutive days. Even one day after seeding, organoids started to compact, and by day 2 they formed clearly compacted organoids. Further compaction continued on days 3 and 4 (FIG. 1C). These organoids could be cultured for up to 9 weeks (D63) while still maintaining compacted organoid morphology with clear tubular structures (FIG. 1D). After 9 weeks in culture, however, they started to collapse. Measurement of the area of the organoids indicated that they continued to grow in size during the entire 9-week culture period (FIG. 1E).

### Example 3: Characterising Gene Expression of Testicular Organoids Cultured on Transwell Inserts in Defined Media

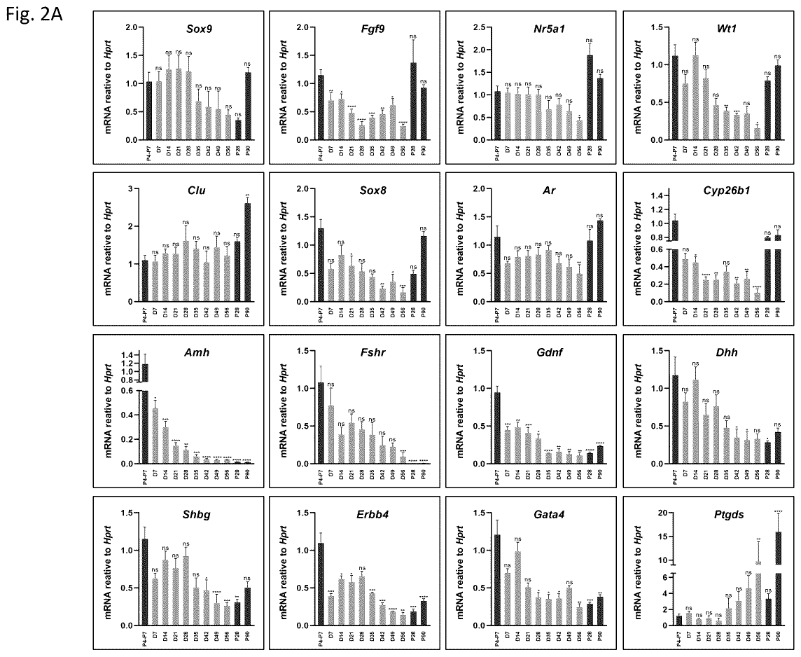

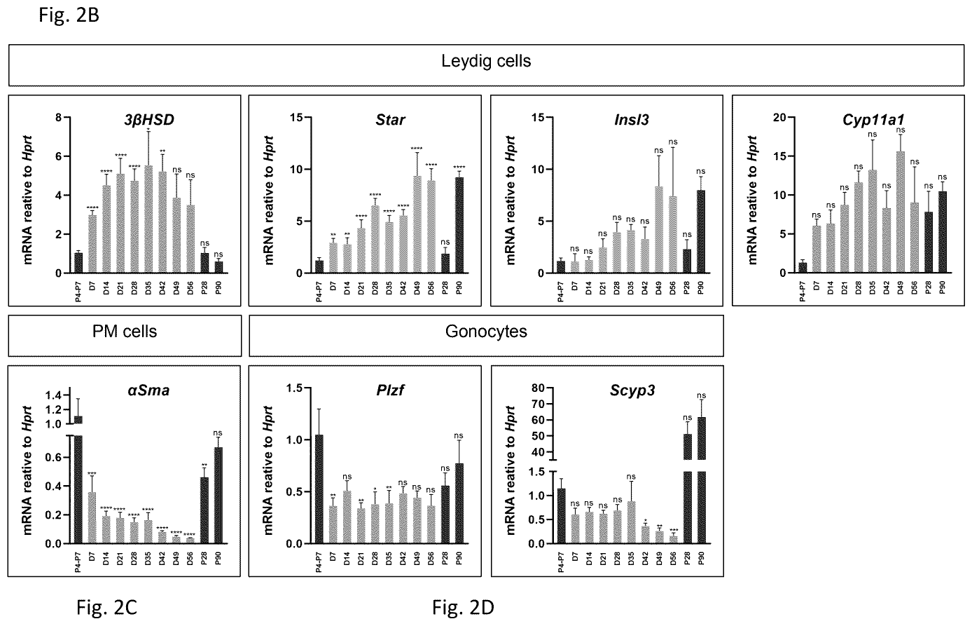

Gene expression profiles of the organoids were assessed and compared to in vivo testis, first focusing on Sertoli-specific markers, as Sertoli cells are the organising hub of the testis. Gene expression was measured at various stages of the 8-week organoid culture period, compared to P4-P7 testis, as the reference point from which the organoids were generated, P28 testis, which correspond to the endpoint, D21 organoids, and the adult testis at P90. As shown in FIG. 2A, many of the Sertoli-specific markers, i.e. Sox9, Nr5a1, Wt1, Clu and Ar displayed similar levels of gene expression in the organoid culture as that of the in vivo testis throughout the 8-week culture. Interestingly, some Sertoli markers varied in expression in neonatal testis versus adult testis; Amh, Fshr, Gdnf Gata4, and others, decreased in expression as the testis matured, while other genes, like Ptgds, showed the opposite pattern. The culture of organoids over the 8-week period mimicked these expression trends, presenting decreased gene expression in Amh, Fshr, Gdnf Gata4 and increased expression of Ptgds over time, as shown in FIG. 2A.

Since the organoid system was composed of an entire testicular cell mixture, and not only Sertoli cells, expression of several markers of other gonadal cell types were also analysed (FIGS. 2B-2D). As for Leydig cell markers (FIG. 2B), while 3βHsd expression was significantly higher than that of in vivo testis, other Leydig markers as Star, Ins13 and Cyp11a1 exhibited levels that are more comparable to the in vivo testis. This indicated that Leydig cells were present and well maintained throughout the 8-week culture period. α-Sma, a marker of PM cells, was also expressed, as in in vivo testes (FIG. 2C). Finally, analysis of two gonocytes markers, Plzf and Scyp3, which are expressed in meiotic cells, indicated that they are also expressed in the organoid system, as shown in FIG. 2D.

Collectively, the gene expression analysis indicates that organoid culture on transwell devices with defined media can support prolonged culture and survival of multiple testicular cell types and maintain gene expression profiles that resemble the in vivo testis.

### Example 4: Testicular Organoids Cultured on Transwell Inserts Organize into Tubular Structures Reminiscent of the Testis

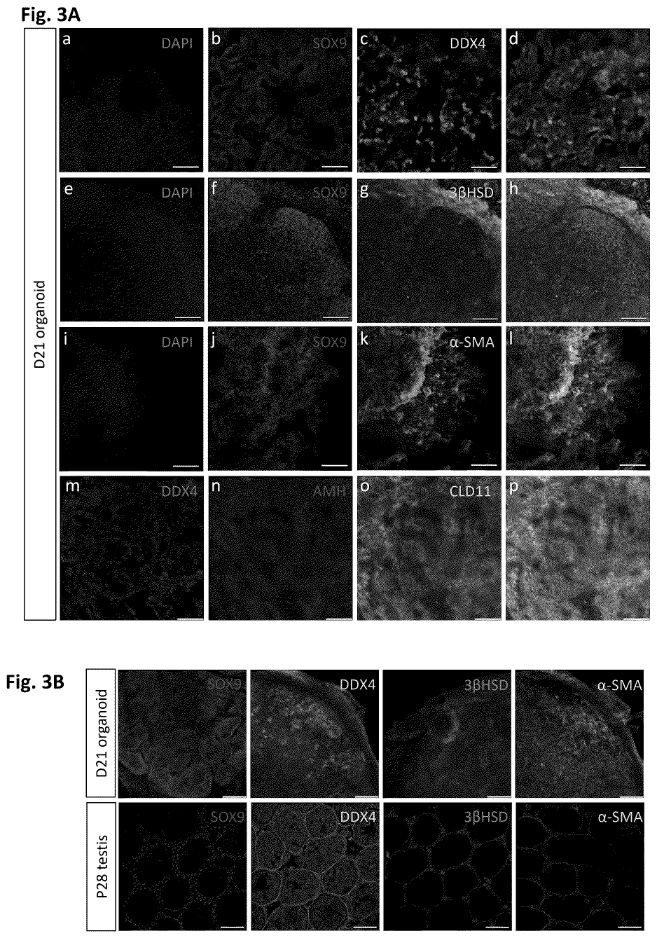

The testes are composed of two main compartments, namely the testis cords that later become the seminiferous tubules and contain Sertoli cells and gonocytes, and the interstitial area, composed mostly of Leydig and PM cells [5, 39]. To explore whether all major testicular cell types are present in the organoids and determine their spatial organisation, day 21 (D21) organoids were immunostained with various markers of the different cell types (FIG. 3A). Immunostaining of whole-mount D21 organoids with SOX9, AMH, CLD11 (Sertoli cells), 3BHSD (Leydig cells), DDX4 (gonocytes) and α-SMA (PM cells) indicated that all major testicular cell types are present within the organoids for a period of 21 days in culture (FIG. 3A). As the organoids are spherical in shape, multiple z-stack images of whole-mount-stained organoids were acquired, generating movies of these organoids with the various co-staining. Remarkably, the organoids presented with spatial organisation that closely resembled the in vivo testis. Sertoli cells formed numerous tubular structures and the DDX4 positive cells were located next to Sertoli cells and within the tubules. Apparently, the gonocytes had a tendency of localizing towards the outer side of the tubules, but adjacent to Sertoli cells (FIG. 3A, panels a-d,). 38HSD-positive Leydig cells were located outside of the SOX9-positive tubules, in an area that corresponds to the interstitial area (FIG. 3A, panels e-h). α-SMA-positive PM cells were located close to Sertoli cells (FIG. 3A, panels i-1). Expression of AMH and CLD11 was strong and indicated the presence of tubular structures composed of Sertoli cells, and containing DDX4-positive germ cells (FIG. 3A, panels m-p).

To better analyze the spatial organisation of testicular organoids and compare it to that of in vivo testis, whole-mount immunostaining of D21 organoids was performed and compared to immunostaining of P28 testis (D21 organoid corresponds to P28 as it was harvested at P7 and cultured in vitro for 21 days). The results are presented in FIG. 3B. Staining with SOX9 Sertoli cell marker clearly showed the formation of tubules, similar to these of the testis. Analysis of movies composed of multiple z-stacks of whole-mount organoids confirmed the formation of tubules by both SOX9 and DAPI staining. DDX4, which marks gonocytes, was strongly expressed and gonocytes seemed to aggregate together. 313HSD immunostaining indicated localization outside of tubules, similar to in vivo testis. α-SMA was strongly expressed in the organoids.

Collectively, the results indicate that the in vitro testicular organoids form compartments and structures that closely resemble the structure of the in vivo testis, and preserve all major cell types for prolonged periods of time.

### Example 5: Generation of Testicular Organoids from Embryonic Testis

In order to obtain organoids from embryonal tests, the same methods established for neonatal testis organoids were used for generation of testis organoids from embryonic gonads.

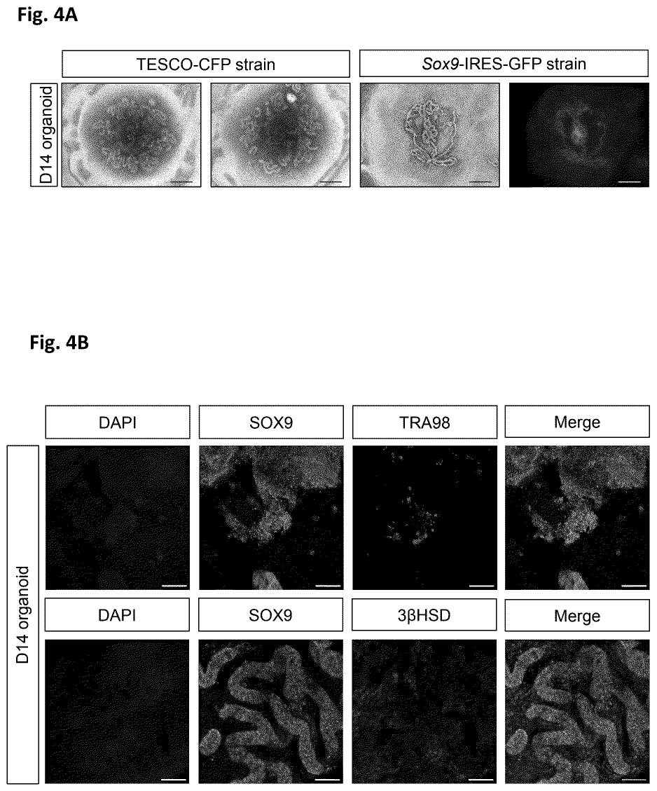

To this aim, testes were harvested from E12.5-E14.5 embryos of either TESCO-CFP or Sox9-IRES-GFP mouse strains, dissociated into single cells and seeded on transwell inserts in defined media. Embryonic-derived testicular cells formed organoids with very pronounced and clear tubular structures, much clearer than these observed using neonatal testis. FIG. 4A presents three different embryonic testis organoids, after 14 days in culture. The right-hand panel displays the GFP fluorescence from organoid derived from the Sox9-IRES-GFP strain. GFP was present within the tubules, indicating that the tubules are formed from Sertoli cells (FIG. 4A).

To better characterize the embryonic testicular organoids that were cultured in vitro for 14 days, whole mount D14 organoids were immune-stained for markers of various gonadal cell types (FIG. 4B). Organoids were strongly stained for SOX9; these SOX9-positive cells formed the tubules. TRA98, which marks gonocytes, was also expressed, indicating the presence of germ cells. Furthermore, 313HSD was expressed and predominantly located outside of the SOX9-positive tubules, within the interstitium, as expected of Leydig cells.

These results indicate that the transwell system, coupled with defined media, is suitable for the formation of both embryonic and neonatal testicular organoids that preserve most gonadal cell types for prolonged periods and highly resemble testicular compartments and structures.

### Example 6: Media Compositions to Support the Immature and Mature States of Sertoli Cells

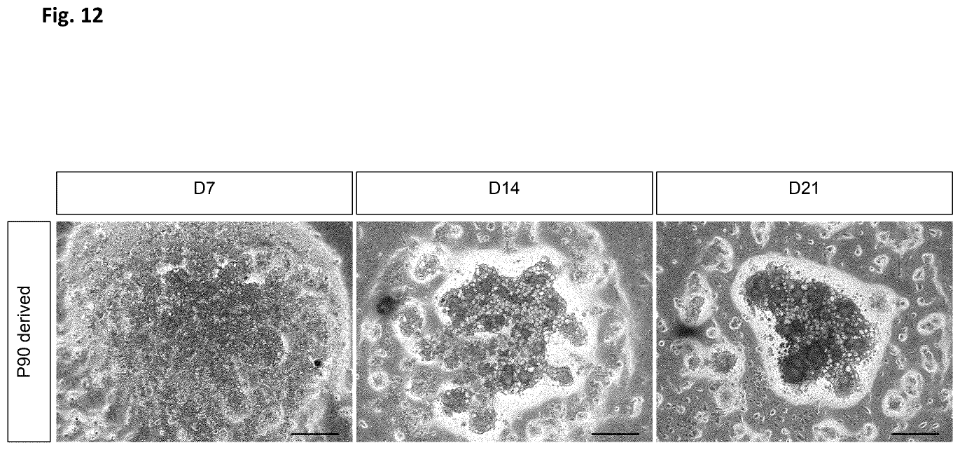

Having shown that the transwell system can support formation of embryonic and neonatal testis organoids, it was sought to also culture mature testis in these conditions. Testicular cells were harvested from P90 mature testis and seeded on transwell inserts. In contrast to the embryonic and neonatal testis, adult testicular cells were not capable of forming organoids, as shown in FIG. 12.

As an alternative approach, it was attempted to mature neonatal testis organoids to adult testis state using defined media modification.

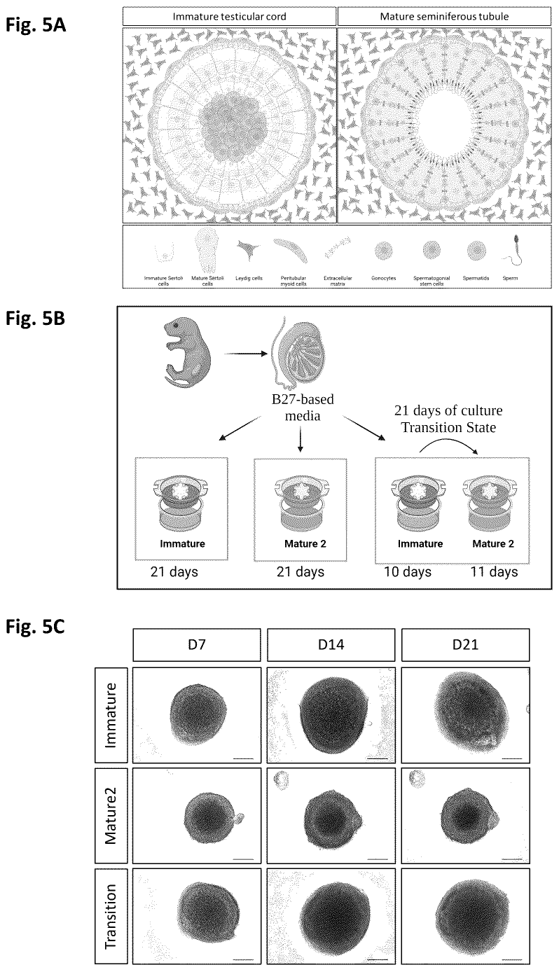

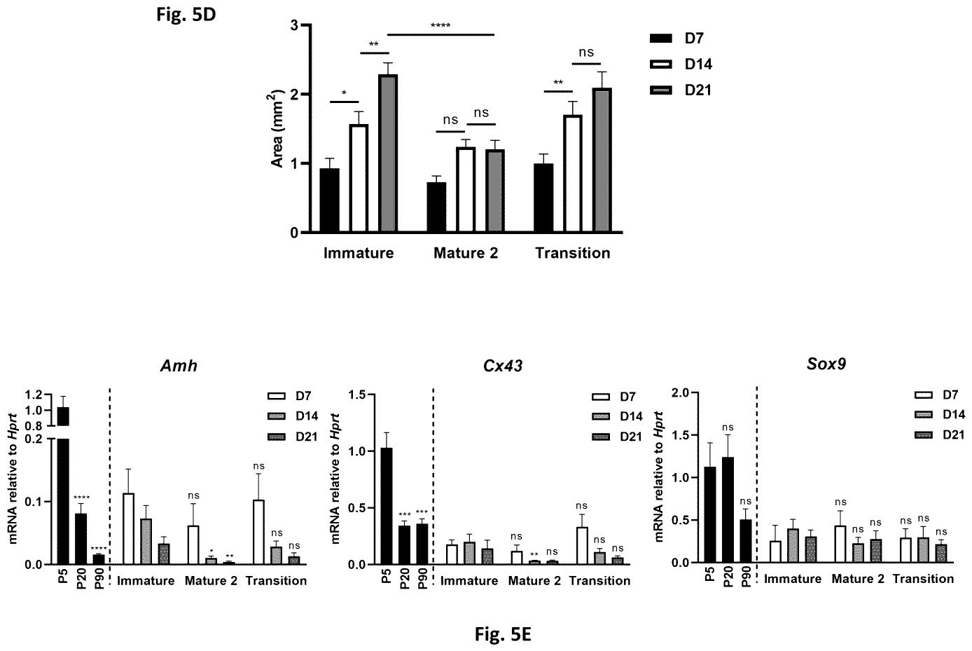

At embryonic stages, the testis is composed of testis cords containing gonocytes, surrounded by immature Sertoli cells and peritubular myoid cells. As shown in the illustration presented in FIG. 5A, Immature Sertoli cells have native capacity to proliferate. At puberty, gonocytes migrate towards the basal lamina and become SSC. Sertoli cells undergo maturation, which promotes their polarization and establishment of the blood-testis barrier. The large polarized Sertoli cells contact spermatids of different stages as the latter undergo meiosis.

Factors that maintain and enhance the immature state include FSH, Insulin like Growth Factor I (IGF-1), Activin A and FGF9. Factors that promote the mature Sertoli state include Androgens, Thyroid Hormones (TH) and Retinoic Acid (RA).

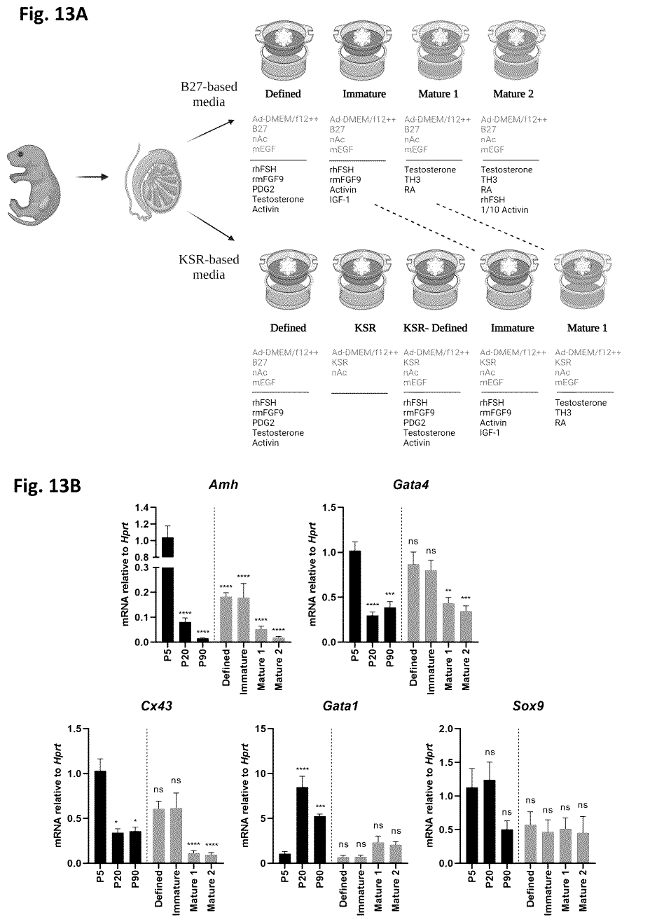

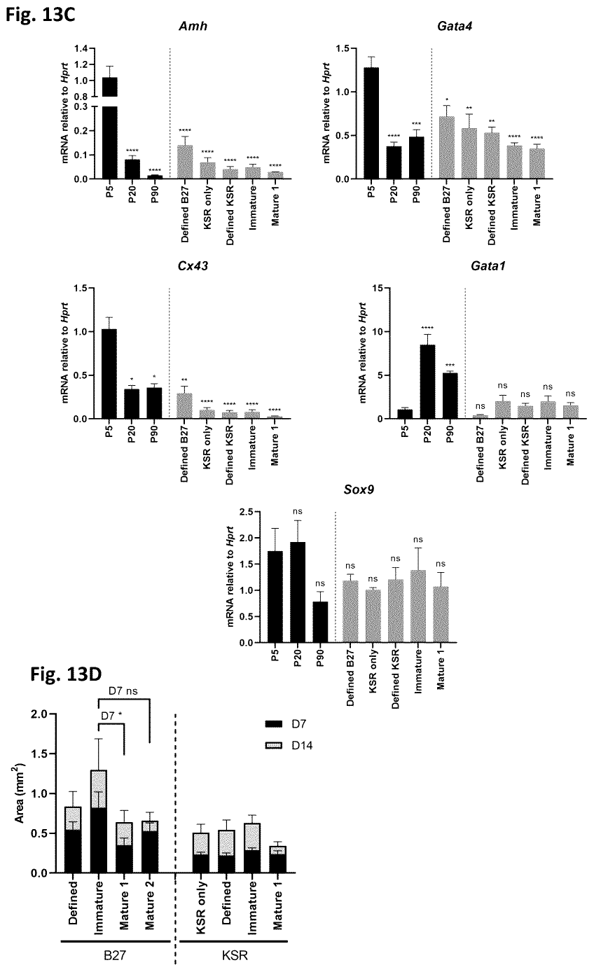

Accordingly, three different media compositions were used: one to support the maintenance of the immature Sertoli state (Immature medium), and two alternative media to promote the maintenance of mature Sertoli cells (Mature 1 and Mature2 media). The various media compositions were tested on either B27- or Knockout serum replacement (KSR)-based media. The media compositions are detailed in Table 2. The experimental outline is illustrated in FIG. 13A. All media compositions were compared to the original defined media that was used throughout the study (contains B27, Table 2).

To test the ability of the various media compositions to promote immature versus mature Sertoli state, the expression levels of Sertoli markers that differ between the immature and mature states were measured using quantitative PCR (qPCR). To verify that the tested markers are indeed more highly expressed in one state over the other, their expression was first assessed in P5, P20 and P90 whole testis. Amh, Gata4 and Cx43 are significantly higher at P5 testis compared to adult testis, i.e., mark the immature Sertoli state, while Gata1 is more highly expressed in adult testis and marks mature Sertoli cells (FIGS. 13B-13C). Sox9 was not significantly altered between the two states and served as a control. Analysis of the expression levels of Amh, Gata4 and Cx43 in organoids cultured for 14 days in defined, immature, or two types of mature media, all containing B27, indicated that the immature media allowed for higher expression of all three immature state markers, while the two mature media led to significantly lower expression levels. In contrast, analysis of Gata1 expression resulted in higher expression in both mature media compared to the immature and defined media. This indicates that the immature media containing B27 is able to promote the immature Sertoli cell state, while the mature media containing B27 are better at promoting the mature Sertoli cell state (FIG. 13B).

Similar analysis was conducted for organoids grown for 14 days in defined media (with B27), KSR only (no growth factors added), defined KSR, immature and mature media with KSR. Unlike the changes observed in expression of the various markers between the immature and mature media based on B27, no significant changes in expression of Amh, Gata4, Cx43 and Gata1 were observed when different combinations of KSR-based media were used (FIG. 13C). This indicates that the addition of KSR to the media masks the effect of the growth factors added to the media to promote the different cell states.

Next, the size of organoids grown for 14 days with the various B27-based and the KSR-based media compositions was determined. In line with what was seen by qPCR, the immature B27-based media was able to promote the immature state, resulting in significantly larger organoids at D7 and D14, while the two mature B27-based media led to smaller size organoids at D7 and D14 (FIG. 13D, left). In contrast, no major change in organoid size was observed when organoids were cultured on KSR-based media on either D7 or D14 (FIG. 13D, right). Based on the results, it was decided to utilize the B27-based media.

In vivo, the testis remains in their immature state until about day P15, after which there is a transition to the mature Sertoli state and spermatogenesis commences. Accordingly, a protocol was devised in which P5 neonatal testes were harvested, cultured in immature media for 10 days (to mimic the P15 stage), and then transferred to the mature media for an additional 11 days, for a total of 21 days in culture (FIG. 5B, the “transition state”). As a control, organoids were cultured for 21 days in either immature media or mature media alone.

As a first read-out, the size of organoids was measured at D7, D14 and D21 of culture. Organoids grown only in immature media exhibited a significant growth in size from D7 to D14 and from D14 to D21 (FIGS. 5C and 5D). In contrast, organoids cultured in mature2 media did not exhibit any growth in size between D7-D21. Interestingly, organoids in the transition media exhibited a significant growth in size from D7 to D14, when they were cultured mainly with immature media, and then showed no significant growth in size from D14 to D21, when they were grown in mature 2 media (FIGS. 5C-D).

Next, Amh, Cx43 and Sox9 expression levels were analysed from organoids cultured in the various media for a period of 7, 14 and 21 days. There was no significant change in gene expression for all genes examined at D7 (FIG. 5E). At D14, however, expression levels of Amh and Cx43 were significantly lower in organoids grown on mature2 media, but not in organoids grown on transition media. On day 21, expression levels of Amh and Cx43 appeared lower in organoids cultured in the mature2 and transition media compared to the immature media. No change was evident in the expression of Sox9, as expected, as this gene does not change much between the immature and mature Sertoli cell states (FIG. 5E).

Collectively, the results indicate that the immature and mature states of Sertoli cells can be maintained and promoted in organoids by manipulating the media compositions and growth factors added to the media.

### Example 7: Analysis of the Transition Media Over Prolonged In Vitro Culture Period

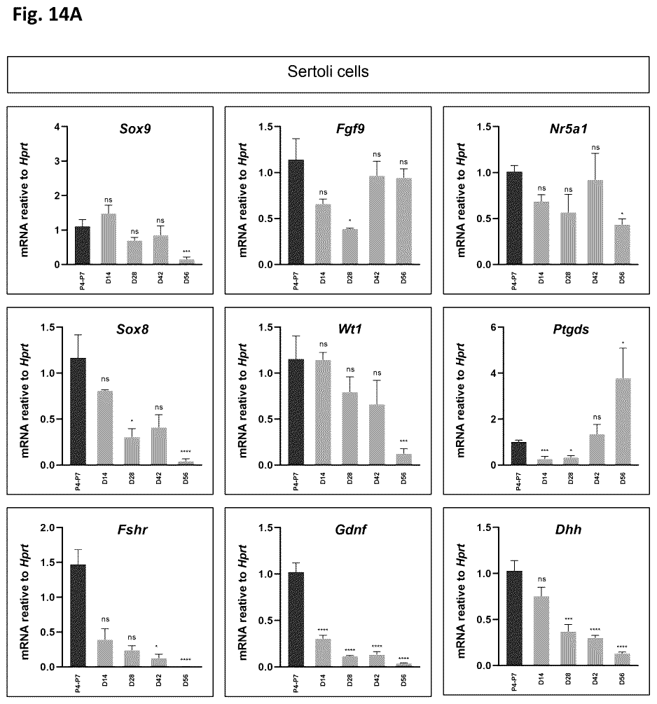

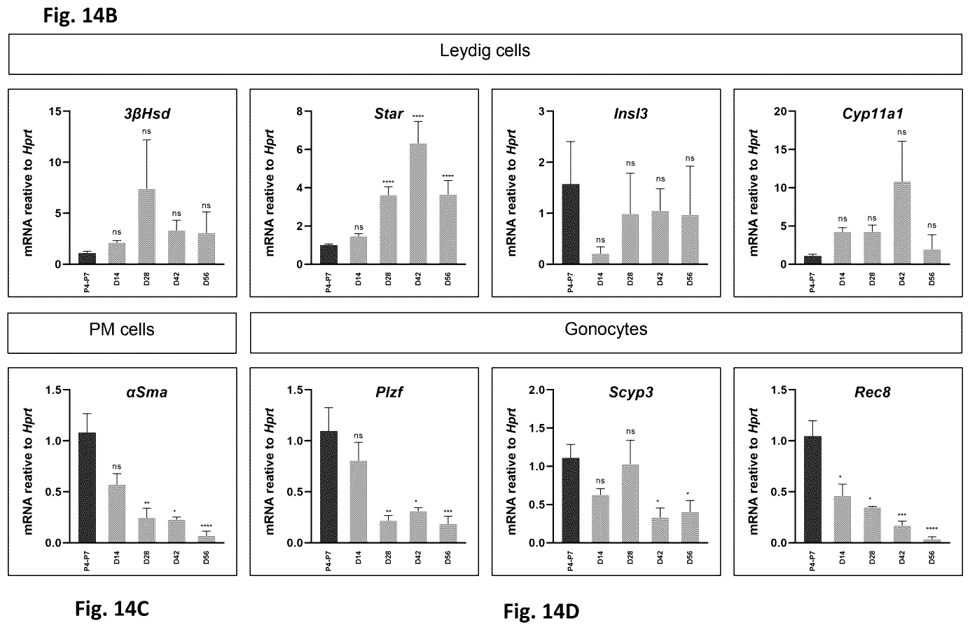

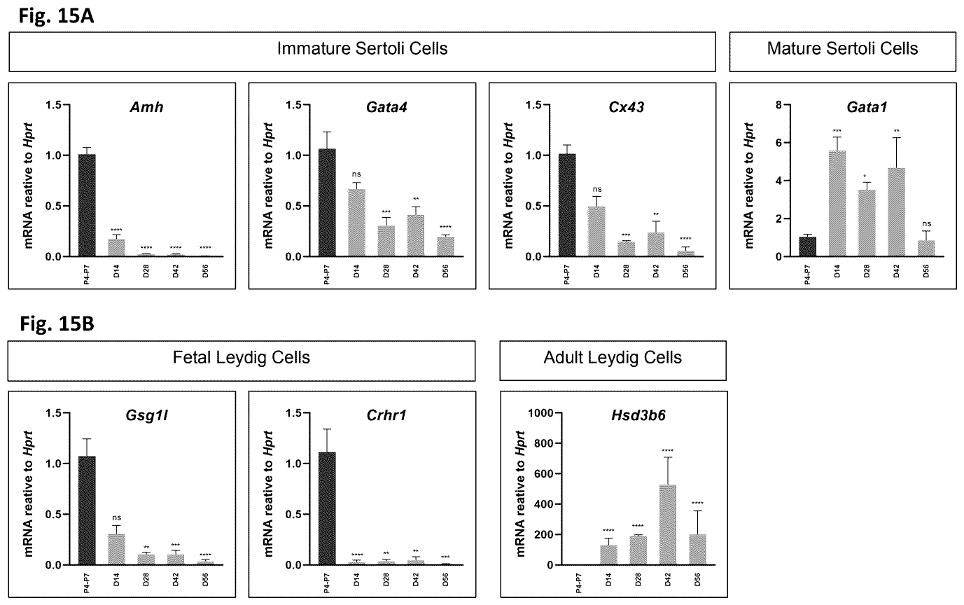

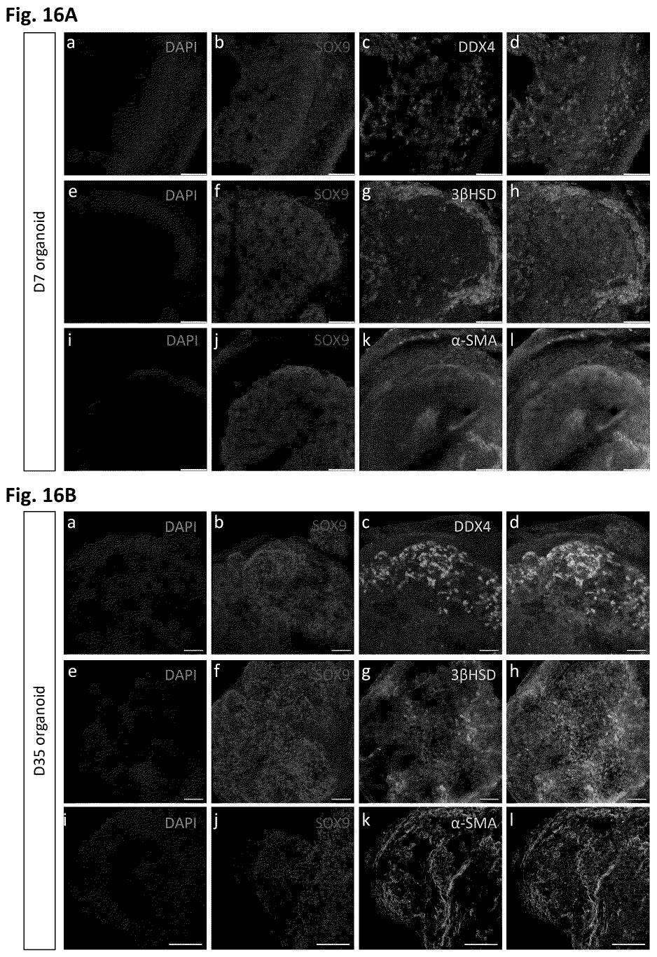

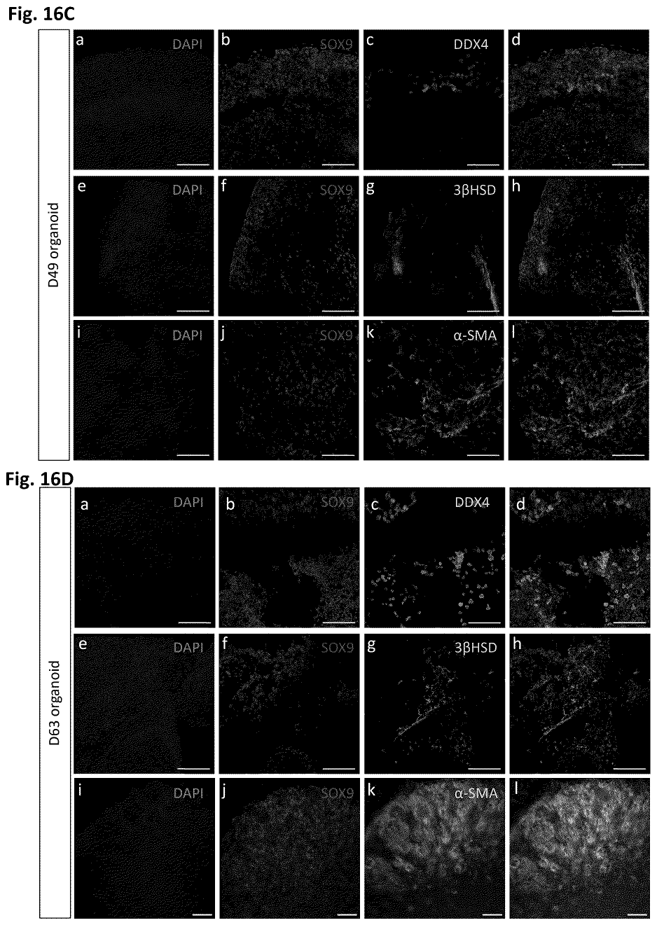

As the transition media combination seemed favourable and more closely mimics the in vivo state, expression analysis (collectively shown in FIGS. 14A-D and FIGS. 15A-B), as well as immunostaining on organoids cultured for up to 9 weeks (D63) on transition media (shown in FIGS. 16A-D).

Expression analysis indicated that most Sertoli markers behave similar to what would be expected from in vivo testis (FIGS. 14A-D and FIGS. 2A-2B). Markers of Leydig cells, PM cells, as well as gonocytes, were also well maintained. Under the transition media, immature Sertoli markers, such as Amh, Gata4 and Cx43 decreased with culture time, while Gata1, which marks mature Sertoli cells, increased in culture compared to P4-P7 testis, as shown in FIG. 15A.

Similar to the maturation of Sertoli cells in the testis, Leydig cells also undergo maturation as testis mature [44]. While immature testis contains Fetal Leydig cells (FLC), these are replaced in adult testis with Adult Leydig cells (ALC). To examine if the transition media also promote maturation from FLC to ALC, expression of genes that were shown to be FLC-specific or ALC-specific were tested. Among the FLC-specific genes are the Gsg11 and Crhr1 while an ALC-specific gene is the Hsd3b6 gene [44].

qPCR analysis indicated that the two FLC markers were markedly decreased in culture already by D14 (in which the organoids are already in mature media), while a major increase is seen with the expression of Hsd36b, which marks ALC (FIG. 15B). These results indicate that the transition media is able to induce maturation also of Leydig cells and not only Sertoli cells.

Next, since the organoids can be maintained for 9 weeks in vitro and all staining were performed on D21 organoids cultured on defined media, staining with major gonadal cell type markers was performed on organoids grown on transition media at D7, D35, D49 and D63, as shown in FIGS. 16A-D).

The staining results indicate that all gonadal cell types and testicular structures are well maintained for the 9-week culture period.

### Example 8: Testicular Organoids Allow Entry of SSC to Meiosis

One of the main functions of the testis is to support spermatogenesis and haploid sperm production. Spermatogenesis is a long process in which diploid spermatogonial stem cells undergo meiosis to form haploid round spermatids that will undergo spermiogenesis, giving rise to the sperm in its mature form with the acrosome cap on the anterior region of the head. One of the hallmarks of meiosis is formation of double strand breaks (DSB) and homologous recombination of genetic material between sister chromatids [45].

Thus, to determine whether the organoids support entry of spermatogonial stem cells into meiosis, D21 organoids, first cultured on defined media, were co-stained with the meiotic marker γH2AX, which labels DSB, and DDX4, which marks all stages of SSC and spermatogonia. γH2AX foci are normally present in intermediate and type B spermatogonia and in preleptotene to zygotene spermatocytes. Type A spermatogonia and round spermatids also stain for γH2AX, but as homogeneous nuclear staining [46, 47].

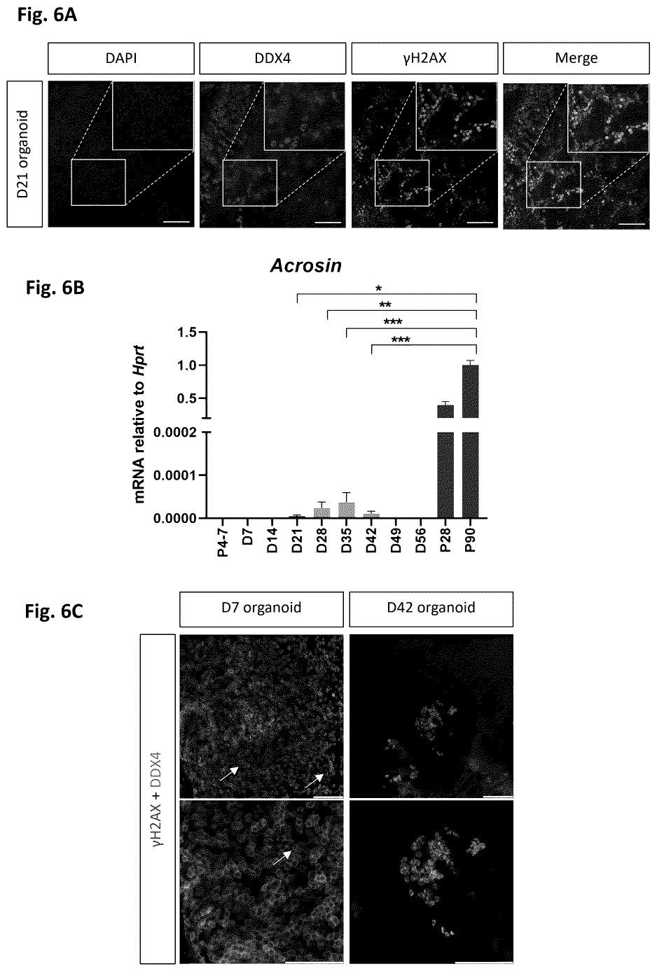

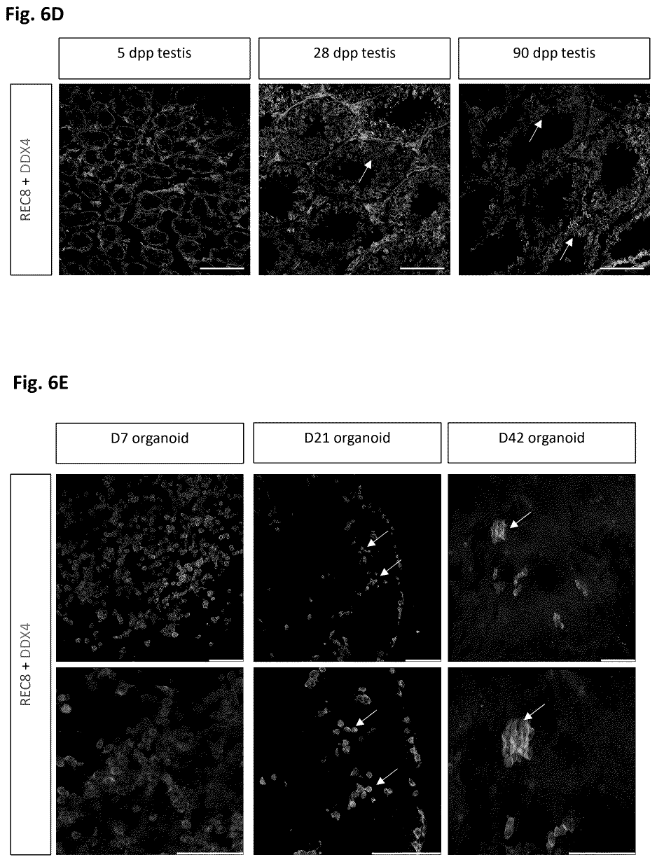

The results presented in FIG. 6A demonstrate that some of the DDX4-positive cells are also γH2AX-positive, indicating that cells may enter meiosis within the organoids in vitro.

Another indication for the presence of meiosis came from qPCR analysis for the expression of Acrosin, a gene specifically expressed at the mature sperm head within the acrosome. While P4-P7 testes do not show any Acrosin expression, as meiosis has yet to commence at that stage, P28 and P90 testes demonstrate very high levels of Acrosin expression (FIG. 6B). Interestingly, in vitro testis organoids exhibited low levels of Acrosin expression, mostly at D21-D42. Although this expression level is very low compared to in vivo testis, its time-dependent presence indicates the existence of small quantities of fully mature sperm at the later stages of organoid culture.

Next, it was analysed whether D7 and D42 organoids, cultured on transition media, also present co-staining of DDX4 and γH2AX. As shown in FIG. 6C, while few cells exhibit co-staining at D7 organoids, many are co-stained in D42 organoids.

Although γH2AX marks spermatogonia, it can also mark cells with DSB, hence, to verify that organoid SSC indeed enter meiosis, the presence of REC8, a subunit of the cohesin complex which function during meiosis [48, 49] was analysed.

First, testis sections of 5, 28, and 90 dpp testis were stained with REC8 and DDX4 antibodies. As expected, no REC8 expression is seen in 5 dpp testis, but substantial REC8 can be seen in 28 dpp and 90 dpp testis section is specific seminiferous tubules, confirming the specificity of the antibody (FIG. 6D). Next, whole mount staining with REC8 and DDX4 was performed on D7, D21 and D42 organoids, cultured in transition media. While no overlap is evident at D7 organoids, double positive cells can be seen in D21 and D42 organoids.

Collectively, the results indicate that in vitro organoid culture can support entry of SSC into meiosis, where some even complete spermatogenesis and form a sperm head containing Acrosin.

### REFERENCES

ADDIN EN.REFLIST 1. Wilhelm D, Palmer S, Koopman P. Sex determination and gonadal development in mammals. Physiol Rev. 2007; 87: 1-28.
2. Rotgers E, Jorgensen A, Yao H H. At the Crossroads of Fate-Somatic Cell Lineage Specification in the Fetal Gonad. Endocr Rev. 2018; 39: 739-59.
3. Nef S, Stevant I, Greenfield A. Characterizing the bipotential mammalian gonad. Curr Top Dev Biol. 2019; 134: 167-94.
4. Gonen N, Lovell-Badge R. The regulation of Sox9 expression in the gonad. Curr Top Dev Biol. 2019; 134: 223-52.
5. Maatouk D M, Capel B. Sexual development of the soma in the mouse. Curr Top Dev Biol. 2008; 83: 151-83.
6. Fayomi A P, Orwig K E. Spermatogonial stem cells and spermatogenesis in mice, monkeys and men. Stem Cell Res. 2018; 29: 207-14.
7. Bashamboo A, Eozenou C, Rojo S, McElreavey K. Anomalies in human sex determination provide unique insights into the complex genetic interactions of early gonad development. Clin Genet. 2017; 91: 143-56.
8. Delot E C, Vilain E. Towards improved genetic diagnosis of human differences of sex development. Nat Rev Genet. 2021; 22: 588-602.
9. Organization W H. Infertility prevalence estimates, 1990-2021. Licence: CC BY-NC-SA 30IGO.2023.
10. Tuttelmann F, Ruckert C, Ropke A. Disorders of spermatogenesis: Perspectives for novel genetic diagnostics after 20 years of unchanged routine. Med Genet. 2018; 30: 12-20.
11. Buganim Y, Itskovich E, Hu Y C, Cheng A W, Ganz K, Sarkar S, et al. Direct reprogramming of fibroblasts into embryonic Sertoli-like cells by defined factors. Cell Stem Cell. 2012; 11: 373-86.
12. Beverdam A, Wilhelm D, Koopman P. Molecular characterization of three gonad cell lines. Cytogenet Genome Res. 2003; 101: 242-9.
13. Bashamboo A, Eozenou C, Jorgensen A, Bignon-Topalovic J, Siffroi J P, Hyon C, et al. Loss of Function of the Nuclear Receptor NR2F2, Encoding COUP-TF2, Causes Testis Development and Cardiac Defects in 46,XX Children. Am J Hum Genet. 2018; 102: 487-93.
14. Miyado M, Inui M, Igarashi M, Katoh-Fukui Y, Takasawa K, Hakoda A, et al. The p.R92W variant of NR5A1/Nr5a1 induces testicular development of 46,XX gonads in humans, but not in mice: phenotypic comparison of human patients and mutation-induced mice. Biol Sex Differ. 2016; 7: 56.
15. Lancaster M A, Knoblich J A. Organogenesis in a dish: modeling development and disease using organoid technologies. Science. 2014; 345: 1247125.
16. Lara N, Sakib S, Dobrinski I. Regulation of Cell Types Within Testicular Organoids. Endocrinology. 2021; 162.
17. Oliver E, Stukenborg J B. Rebuilding the human testis in vitro. Andrology. 2020; 8: 825-34.
18. AbuMadighem A, Shuchat S, Lunenfeld E, Yossifon G, Huleihel M. Testis on a chip-a microfluidic three-dimensional culture system for the development of spermatogenesisin-vitro. Biofabrication. 2022; 14.
19. Alves-Lopes J P, Soder O, Stukenborg J B. Testicular organoid generation by a novel in vitro three-layer gradient system. Biomaterials. 2017; 130: 76-89.
20. Baert Y, De Kock J, Alves-Lopes J P, Soder O, Stukenborg J B, Goossens E. Primary Human Testicular Cells Self-Organize into Organoids with Testicular Properties. Stem Cell Reports. 2017; 8: 30-8.
21. Edmonds M E, Woodruff T K. Testicular organoid formation is a property of immature somatic cells, which self-assemble and exhibit long-term hormone-responsive endocrine function. Biofabrication. 2020; 12: 045002.
22. Sakib S, Uchida A, Valenzuela-Leon P, Yu Y, Valli-Pulaski H, Orwig K, et al. Formation of organotypic testicular organoids in microwell culturedagger. Biol Reprod. 2019; 100: 1648-60.
23. Rezaei Topraggaleh T, Rezazadeh Valojerdi M, Montazeri L, Baharvand H. A testis-derived macroporous 3D scaffold as a platform for the generation of mouse testicular organoids. Biomater Sci. 2019; 7: 1422-36.
24. Sekido R, Lovell-Badge R. Sex determination involves synergistic action of SRY and SF1 on a specific Sox9 enhancer. Nature. 2008; 453: 930-4.
25. Furuyama K, Kawaguchi Y, Akiyama H, Horiguchi M, Kodama S, Kuhara T, et al. Continuous cell supply from a Sox9-expressing progenitor zone in adult liver, exocrine pancreas and intestine. Nat Genet. 2011; 43: 34-41.
26. Buzzard J J, Wreford N G, Morrison J R. Marked extension of proliferation of rat Sertoli cells in culture using recombinant human FSH. Reproduction. 2002; 124: 633-41.
27. Gustin S E, Stringer J M, Hogg K, Sinclair A H, Western P S. FGF9, activin and TGFbeta promote testicular characteristics in an XX gonad organ culture model. Reproduction. 2016; 152: 529-43.
28. Wilhelm D, Martinson F, Bradford S, Wilson M J, Combes A N, Beverdam A, et al. Sertoli cell differentiation is induced both cell-autonomously and through prostaglandin signaling during mammalian sex determination. Dev Biol. 2005; 287: 111-24.
29. Malki S, Nef S, Notarnicola C, Thevenet L, Gasca S, Mejean C, et al. Prostaglandin D2 induces nuclear import of the sex-determining factor SOX9 via its cAMP-PKA phosphorylation. EMBO J. 2005; 24: 1798-809.
30. Buzzard J J, Wreford N G, Morrison J R. Thyroid hormone, retinoic acid, and testosterone suppress proliferation and induce markers of differentiation in cultured rat sertoli cells. Endocrinology. 2003; 144: 3722-31.
31. Buzzard J J, Farnworth P G, De Kretser D M, O'Connor A E, Wreford N G, Morrison J R. Proliferative phase sertoli cells display a developmentally regulated response to activin in vitro. Endocrinology. 2003; 144: 474-83.
32. Takasato M, Er P X, Chiu H S, Little M H. Generation of kidney organoids from human pluripotent stem cells. Nat Protoc. 2016; 11: 1681-92.
33. Takasato M, Er P X, Chiu H S, Maier B, Baillie G J, Ferguson C, et al. Kidney organoids from human iPS cells contain multiple lineages and model human nephrogenesis. Nature. 2015; 526: 564-8.
34. Sontheimer-Phelps A, Hassell B A, Ingber D E. Modelling cancer in microfluidic human organs-on-chips. Nat Rev Cancer. 2019; 19: 65-81.
35. Fedele G, Cazzaniga A, Castiglioni S, Locatelli L, Tosoni A, Nebuloni M, Maier J A M. The presence of BBB hastens neuronal differentiation of cerebral organoids—The potential role of endothelial derived BDNF. Biochem Biophys Res Commun. 2022; 626: 30-7.
36. Zhao F, Wang J, Wang Q, Hou Z, Zhang Y, Li X, et al. Organoid technology and lung injury mouse models evaluating effects of hydroxychloroquine on lung epithelial regeneration. Exp Anim. 2022; 71: 316-28.
37. Li P, Li Y, Wang Y, Liu J, Lavrijsen M, Li Y, et al. Recapitulating hepatitis E virus-host interactions and facilitating antiviral drug discovery in human liver-derived organoids. Sci Adv. 2022; 8: eabj5908.
38. Capel B. The role of Sry in cellular events underlying mammalian sex determination. Curr Top Dev Biol. 1996; 32: 1-37.
39. Stevant I, Nef S. Genetic Control of Gonadal Sex Determination and Development. Trends Genet. 2019; 35: 346-58.
40. Meroni S B, Galardo M N, Rindone G, Gorga A, Riera M F, Cigorraga S B. Molecular Mechanisms and Signaling Pathways Involved in Sertoli Cell Proliferation. Front Endocrinol (Lausanne). 2019; 10: 224.
41. Huch M, Dorrell C, Boj S F, van Es J H, Li V S, van de Wetering M, et al. In vitro expansion of single Lgr5+ liver stem cells induced by Wnt-driven regeneration. Nature. 2013; 494: 247-50.
42. Fayaz M A, Honaramooz A. Culture media and supplements affect proliferation, colony-formation, and potency of porcine male germ cells. Theriogenology. 2022; 187: 227-37.
43. Gholami K, Pourmand G, Koruji M, Sadighigilani M, Navid S, Izadyar F, Abbasi M. Efficiency of colony formation and differentiation of human spermatogenic cells in two different culture systems. Reprod Biol. 2018; 18: 397-403.
44. Sararols P, Stevant I, Neirijnck Y, Rebourcet D, Darbey A, Curley M K, et al. Specific Transcriptomic Signatures and Dual Regulation of Steroidogenesis Between Fetal and Adult Mouse Leydig Cells. Front Cell Dev Biol. 2021; 9: 695546.
45. Murakami H, Keeney S. Regulating the formation of DNA double-strand breaks in meiosis. Genes Dev. 2008; 22: 286-92.
46. Hamer G, Roepers-Gajadien H L, van Duyn-Goedhart A, Gademan I S, Kal H B, van Buul P P, de Rooij D G. DNA double-strand breaks and gamma-H2AX signaling in the testis. Biol Reprod. 2003; 68: 628-34.
47. Tadokoro Y, Yomogida K, Yagura Y, Yamada S, Okabe M, Nishimune Y. Characterization of histone H2A.X expression in testis and specific labeling of germ cells at the commitment stage of meiosis with histone H2A.X promoter-enhanced green fluorescent protein transgene. Biol Reprod. 2003; 69: 1325-9.
48. Bannister L A, Reinholdt L G, Munroe R J, Schimenti J C. Positional cloning and characterization of mouse mei8, a disrupted allelle of the meiotic cohesin Rec8. Genesis. 2004; 40: 184-94.
49. Biswas U, Hempel K, Llano E, Pendas A, Jessberger R. Distinct Roles of Meiosis-Specific Cohesin Complexes in Mammalian Spermatogenesis. PLoS Genet. 2016; 12: e1006389.
50. Franca L R, Hess R A, Dufour J M, Hofmann M C, Griswold M D. The Sertoli cell: one hundred fifty years of beauty and plasticity. Andrology. 2016; 4: 189-212.
51. Cool J, DeFalco T J, Capel B. Vascular-mesenchymal cross-talk through Vegf and Pdgf drives organ patterning. Proc Natl Acad Sci USA. 2011; 108: 167-72.
52. Potter S J, DeFalco T. Role of the testis interstitial compartment in spermatogonial stem cell function. Reproduction. 2017; 153: R151-R62.
53. Low J H, Li P, Chew E G Y, Zhou B, Suzuki K, Zhang T, et al. Generation of Human PSC-Derived Kidney Organoids with Patterned Nephron Segments and a De Novo Vascular Network. Cell Stem Cell. 2019; 25: 373-87 e9.
54. Pham M T, Pollock K M, Rose M D, Cary W A, Stewart H R, Zhou P, et al. Generation of human vascularized brain organoids. Neuroreport. 2018; 29: 588-93.
55. Oakberg E F. Duration of spermatogenesis in the mouse and timing of stages of the cycle of the seminiferous epithelium. Am J Anat. 1956; 99: 507-16.
56. Hogg K, Western P S. Differentiation of Fetal Male Germline and Gonadal Progenitor Cells Is Disrupted in Organ Cultures Containing Knockout Serum Replacement. Stem Cells Dev. 2015; 24:2899-911.
57. Clevers H. Modeling Development and Disease with Organoids. Cell. 2016; 165: 1586-97.
58. Hayashi K, Ohta H, Kurimoto K, Aramaki S, Saitou M. Reconstitution of the mouse germ cell specification pathway in culture by pluripotent stem cells. Cell. 2011; 146: 519-32.
59. Hayashi K, Saitou M. Generation of eggs from mouse embryonic stem cells and induced pluripotent stem cells. Nat Protoc. 2013; 8: 1513-24.
60. Gonen N, Eozenou C, Mitter R, Elzaiat M, Stevant I, Aviram R, et al. In vitro cellular reprogramming to model gonad development and its disorders. Sci Adv. 2023; 9: eabn9793.
61. Knarston I M, Pachernegg S, Robevska G, Ghobrial I, Er P X, Georges E, et al. An In Vitro Differentiation Protocol for Human Embryonic Bipotential Gonad and Testis Cell Development. Stem Cell Reports. 2020; 15: 1377-91.
62. Yoshino T, Suzuki T, Nagamatsu G, Yabukami H, Ikegaya M, Kishima M, et al. Generation of ovarian follicles from mouse pluripotent stem cells. Science. 2021; 373: eabe0237.
63. Mottram R, Feltbower R, Hayden C, Lane S, Mitchell R, Anderson R, Glaser A. Fertility and reproductive health after ovarian and testicular tissue storage: The United Kingdom Registry Study of young people (UKSTORE). Archives of Disease in Childhood. 2023; 108: A288-A9.
64. Strange D P, Zarandi N P, Trivedi G, Atala A, Bishop C E, Sadri-Ardekani H, Verma S. Human testicular organoid system as a novel tool to study Zika virus pathogenesis. Emerg Microbes Infect. 2018; 7: 82.
65. Saitou M, Miyauchi H. Gametogenesis from Pluripotent Stem Cells. Cell Stem Cell. 2016; 18: 721-35.

The invention claimed is:

1.-59. (canceled)

60. A testis organoid comprising primary testis cells obtained from neonatal and/or embryonic testis, wherein the cells form a three dimensional structure on a transwell insert in the presence of a defined culture media, wherein the defined culture media is a serum-free media, comprising B-27 supplement and one or more of: Epidermal Growth Factor (EGF), Follicular stimulating hormone (FSH), Fibroblast Growth Factor 9 (FGF9), Prostaglandin D2 (PDG2), Testosterone and Activin A, said organoid is viable for at least three weeks in culture and capable of expressing one or more cellular markers at a spatial and/or temporal pattern similar to that of an in-vivo testis.

61. The testis organoid according to claim 60, wherein the testis cells comprise Sertoli cells, Leydig cells, peritubular myoid (PM) cells and gonocytes.

62. The testis organoid according to claim 60, viable for at least 7 weeks in culture.

63. The testis organoid according to claim 60, wherein the organoid comprises tubular-like structures comprising Sertoli cells and interstitial regions comprising Leydig cells, located externally to said tubular structure, after 7 or more days in culture, wherein the tubular-like structures are organized spatially to mimic seminiferous tubules of in vivo testis.

64. The testis organoid according claim 60, wherein said organoid is capable of expressing acrosin, after at least 7 days in culture.

65. The testis organoid according claim 60, wherein at least some spermatogonial stem cells (SSC) of the organoid enter meiosis, after at least 7 days in culture.

66. The testis organoid according to claim 60, wherein the neonatal cells are obtained from neonatal mice, aged between postnatal days 4-7 (P4-P7) and/or wherein the embryonal cells are obtained from mice embryos on embryonic days E 12.5-14.5.

67. A method for producing a testis organoid, the method comprising:
a) obtaining primary testis cells from a neonatal and/or embryonic testis;
b) seeding the testis cells on a Transwell insert;
c) culturing the cells in the presence of a defined culture medium comprising B-27 supplement;
wherein the organoid is viable for at least three weeks in culture and is capable of expressing one or more cellular markers at a spatial and/or temporal pattern similar to that of an in-vivo testis.

68. The method according to claim 67, comprising seeding at least about 5×10⁴ testis cells per organoid.

69. The method according to claim 67, wherein the testis cells are obtained by mechanical and/or enzymatic dissociation of the testis.

70. The method according to claim 67, wherein the testis cells comprise Sertoli cells, Leydig cells, peritubular myoid (PM) cells and gonocytes.

71. The method according to claim 67, wherein the defined culture media is serum-free media.

72. The method according to claim 67, wherein the defined culture media comprises: Epidermal Growth Factor (EGF), Follicular stimulating hormone (FSH), Fibroblast Growth Factor 9 (FGF9), Prostaglandin D2 (PDG2), Testosterone, Activin A, or any combinations thereof.

73. The method according to claim 72, wherein the EGF is added at a concentration of about 1-100 ng/ml, and/or wherein the FSH is added at a concentration of about 1-100 ng/ml; and/or wherein the FGF9 is added at a concentration of about 1-100 ng/ml; and/or wherein the PDG2 is added at a concentration of about 100-1000 ng/ml; and/or wherein the Testosterone is added at a concentration of about 0.5-2 μM; and/or wherein the Activin is added at a concentration of about 1-100 ng/ml.

74. The method according to claim 67, wherein the neonatal cells are obtained from neonatal mice, aged between postnatal days 4-7 (P4-P7) and/or wherein the embryonal cells are obtained from mice embryos on embryonic days E 12.5-14.5.

75. The method according to claim 67, wherein, after at least 7 days in culture, the organoid comprises tubular-like structures comprising Sertoli cells and interstitial regions comprising Leydig cells, located externally to said tubular structure.

76. The method according to claim 67, wherein the organoid is viable for at least 7 weeks in culture.

77. A method for producing a mature testis organoid, the method comprising:
a) obtaining primary testis cells from a neonatal and/or embryonic testis;
b) seeding the testis cells on a Transwell insert;
c) culturing the cells in the presence of a first culture medium for a first period of time, wherein the first culture medium a serum free medium comprising B27 supplement and further comprises one or more of: Epidermal Growth Factor (EGF), Follicular stimulating hormone (FSH), Fibroblast Growth Factor 9 (FGF9), Activin A, IGF-1;
d) replacing the first cellular medium with a second culture medium wherein the second culture medium is a serum free medium, comprising B27 supplement and further comprises one or more of: Epidermal Growth Factor (EGF), Follicular stimulating hormone (FSH), Testosterone, Activin A, Thyroid hormone, and Retinoic acid; and
d) culturing the cells in the presence of the second culture medium;
wherein the mature organoid is viable for at least three weeks in culture and is capable of expressing one or more cellular markers at a spatial and/or temporal pattern similar to that of an in-vivo testis.

78. The method according to claim 77, wherein, in the presence of the first culture media, increased expression of one or more of the cellular markers Amh, Gata4 and/or Cx43 is facilitated; and/or wherein, in the presence of the second culture media, increased expression of Gata1 is facilitated.

79. The method according to claim 77, wherein the first period of time is in the range of about 4-14 days; and/or wherein after at least 7 days in culture, the organoid comprises tubular-like structures comprising Sertoli cells and interstitial regions comprising Leydig cells, located externally to said tubular structure.
